(19) United States
(12) Patent Application Publication
(10) Pub. No.: US 2014/0309171 A1
(43) Pub. Date: Oct. 16, 2014

(54) MARKER FOR BLOOD COAGULATION

(71) Applicant: Universite Catholique De Louvain, Louvain-la-Neuve (BE)

(72) Inventors: Sandrine Horman, Dion-Valmont (BE); Christophe Beauloye, Dion-Valmont (BE); Marie-Blanche Onselaer, Saint-Leger (BE); Luc Bertrand, Orbais (BE); Jean-Louis Vanoverschelde, WezembeekOppem (BE)

(21) Appl. No.: 14/360,162

(22) PCT Filed: Nov. 21, 2012
(86) PCT No.: PCT/EP2012/073263

§ 371 Date: May 22, 2014

(30) Foreign Application Priority Data
Nov. 22, 2011 (EP) PCT/EP2011/070721

(51) Int. Cl. G01N 33/573

(52) U.S. Cl. CPC G01N 33/573
USPC 514/13.7; 435/7.4; 514/56

(57) ABSTRACT

The invention relates to the use of phosphorylated Acetyl-CoA carboxylase (ACC) in platelets from a subject as a biomarker for blood coagulation, preferably blood coagulation in the subject, preferably encompassing activation of the coagulation cascade as well as platelet activation, preferably thrombin-induced platelet activation. The invention encompasses uses and methods for the diagnosis or the monitoring of blood coagulation in the subject. The invention further relates to uses and methods for screening for anti-coagulants or pro-coagulants.

### FIELD OF THE INVENTION

The invention pertains to the field of biomarkers, particularly markers useful for the evaluation of processes involved in blood coagulation. The invention more particularly relates to uses and methods for the diagnosis or monitoring of blood coagulation in subjects, including screening assays for anti-coagulants or pro-coagulants.

### BACKGROUND OF THE INVENTION

Blood coagulation is a complex process by which blood clots. It is an important part of haemostasis, the process that controls the flow of blood following vascular injury and that includes blood clotting and the subsequent dissolution of the clot following repair of the injured tissue.

Platelet activation and subsequent degranulation and aggregation (clumping together) are known to play a pivotal role in blood coagulation. Platelets become activated through binding to subendothelial proteins such as collagen that are exposed to the bloodstream when the endothelial layer is injured. Activated platelets release the contents of stored granules including, e.g., adenosine diphosphate (ADP) and thromboxane A₂ (TXA₂) into the blood plasma. This in turn activates additional platelets which then aggregate to form a platelet aggregate.

Little is known about a role of adenosine monophosphate (AMP)-activated protein kinase (AMPK) in the regulation of platelet function. Fleming et al (2003. Thromb Haemost. 90: 863-867) reported the presence of AMPK in washed human platelets and its activation by insulin. Randriamboavonjy et al. (2010 Blood 116: 2134-2140) have advanced a role for AMPK in the regulation of platelet function in vitro. In particular, they hypothesised that the α2 AMPK isoform, by affecting Fyn phosphorylation and activity, plays a role in platelet αIIbβ3 integrin signalling, leading to clot retraction and thrombus stability.

Platelets are also activated by thrombin. Thrombin is a serine protease that acts on platelets by activation of a unique class of cell-surface protease activated receptors (PARs) that are members of the large family of G protein-coupled seven transmembrane domain receptors (GPCRs). The prevailing view is that human platelets utilise PAR1 and PAR4 which are coupled to heterotrimeric G_{q} and G₁₂₋₁₃ proteins to initiate signalling cascades leading to increases in cytosolic calcium (Ca²⁺), secretion of autocrine activators, and drastic shape change, which all promote platelet aggregation resulting primarily from activation of the αIIbβ3 fibrinogen receptor. Thrombin also catalyses the last step of the coagulation cascade responsible for the formation of fibrin. Thrombin itself is produced in the coagulation cascade by the enzymatic cleavage of two sites on prothrombin by activated Factor X (Xa).

The coagulation cascade represents the second component of the blood coagulation process. The coagulation cascade involves a series (i.e., cascade) of reactions, in which a zymogen (inactive enzyme precursor) is activated to become an active enzyme that then catalyzes the next reaction in the reaction cascade, ultimately resulting in the formation of a fibrin clot, which strengthens the platelet aggregate. The zymogens are also known as coagulation factors or clotting factors.

A reliable evaluation of the capacity of a subject's blood to coagulate in a timely and effective fashion is crucial to various surgical and medical procedures, such as for example cardiac surgery. Moreover, dependable detection of abnormal blood coagulation can inform the selection of appropriate treatment of patients suffering from haemostasis disorders, including clotting and bleeding disorders. In this connection, it is for example crucial for clinicians to discriminate bleeding related to a haemostasis disorder and bleeding due to a surgical procedure itself.

Whereas several tests are currently available to evaluate the function of the coagulation system, there remains a continuing interest in and need for further alternative and/or improved tests for blood coagulation processes. Moreover, the currently available tests are informative only as to one aspect of the coagulation system, in particular platelet aggregation or coagulation cascade, but do not simultaneously assess platelet activation and activation of the coagulation cascade. In addition, existing tests are performed ex vivo, i.e., after stimulation of a blood sample removed from a subject by a coagulation agonist. Therefore, although the available tests can allow to evaluate for example the capacity of a subject's platelets to respond to a coagulation stimulus in vitro, they generally do not allow to measure the occurrence of actual platelet activation in the subject (i.e., in vivo). Besides the measurement of thromboxane, which, however, is secreted from platelets and is not detected in platelets, there exists no marker allowing to readily evaluate the platelet response in the subject. Furthermore, most available tests do not allow to evaluate agonist-specific platelet activation, e.g., thrombin-induced platelet activation.

Consequently, there exists a continuous need for additional and preferably improved tests that asses the function of the coagulation system. In particular, there remains a need in the art for reliable and simple tests that assess platelet activation, and preferably that can simultaneously assess platelet activation and activation of the coagulation cascade. Also particularly wanted are tests that characterise in vivo platelet activation, in particular thrombin-specific in vivo platelet activation.

### SUMMARY OF THE INVENTION

The present invention addresses one or more of the above discussed needs in the art.

As shown in the experimental section, the inventors have realised that inhibition of Ca²⁺/calmodulin-dependent protein kinase kinase beta (CaMKKbeta) blocked platelet aggregation induced by thrombin, but not adenosine diphosphate (ADP)-induced platelet aggregation. CaMKKbeta inhibition also prevented platelet activation as manifested by the prevention of integrin αIIbβ3 activation and the prevention of α-granules secretion following inhibition of CaMKKbeta. Furthermore, it was shown that thrombin unexpectedly increased Acetyl-CoA carboxylase (ACC) phosphorylation, which could be prevented by inhibiting CaMKKbeta. Said increased ACC phosphorylation in platelets was not observed with the platelet agonists ADP, the thromboxane A2 receptor agonist U46619 or collagen. These in vitro results demonstrate a clear correlation between platelet activation and subsequent platelet degranulation and aggregation specifically induced by thrombin (or specifically induced through activation of thrombin receptor(s) on the surface of platelets) and ACC phosphorylation in platelets.

ACC is a substrate of AMP-activated protein kinase (AMPK) and it is known to be involved in the metabolism of fatty acids by catalysing the carboxylation of Acetyl-CoA to produce malonyl-CoA. Little is known about a role of AMPK beyond metabolism in platelets and a role of ACC beyond metabolism has not been described before in platelets. The usefulness of ACC as a biomarker for platelet aggregation and blood coagulation in vitro is therefore entirely unexpected.

Moreover, the inventors surprisingly found that ACC phosphorylation also represents a valuable biomarker for platelet aggregation and blood coagulation in vivo. Indeed, as also shown in the experimental section, the inventors have found that ACC phosphorylation in platelets from a subject demonstrably correlates with blood coagulation in the subject. This correlation with in vivo coagulation status could not be predicted from the aforementioned in vitro observations, among others because (1) the in vitro studies have been performed with isolated and washed platelets (i.e., substantially without confounding factors), whereas in the in vivo situation interactions with leukocytes, endothelium and the coagulation cascade have to be taken into account and (2) a pharmacological dose of thrombin was applied to the platelets in the in vitro studies, whereas the level of thrombin that is produced in the circulation of a subject (e.g., during post-chirurgical bleeding) is not predetermined but has to be prognosticated. The inventors have also found that ACC phosphorylation in platelets from a subject demonstrably correlates with thrombin generation in the subject.

The invention thus allows to characterise the platelet response and more generally blood coagulation occurring in the patient, which can be conveniently denoted as in vivo. The invention also allows to characterise thrombin generation and more generally activation of the coagulation cascade in vivo.

Hence, phosphorylated ACC represents a useful blood coagulation marker, more particularly a marker for platelet activation specifically by thrombin (or platelet activation specifically induced through activation of thrombin receptor(s) on the surface of the platelets) to be applied for example in pathophysiology studies and in evaluating treatment or anticipating onset of bleeding complications. Phosphorylated ACC also seems useful as a biomarker for thrombin generation and more generally activation of the coagulation cascade.

Given the central role of thrombin in both platelet activation and coagulation cascade, the invention advantageously allows to evaluate platelet activation and activation of the coagulation cascade by a single marker (ACC phosphorylation).

Accordingly, in one aspect, the invention provides the use of phosphorylated Acetyl-coenzyme A carboxylase (Acetyl-CoA carboxylase, ACC) in platelets from a subject as a biomarker for blood coagulation.

In preferred embodiments, the invention provides the use of phosphorylated ACC in platelets from a subject as a biomarker for blood coagulation in the subject. With the term “blood coagulation” is meant herein the process of blood clotting encompassing both platelet activation as well as activation of the coagulation cascade. The term particularly encompasses both “in vivo blood coagulation”, i.e., blood coagulation in (i.e., occurring or taking place inside or within) a subject, as well as “in vitro blood coagulation” or “ex vivo blood coagulation”, i.e., blood coagulation out of (occurring or taking place outside or without) a subject, such as, for example, in a test tube. The phrase “blood coagulation in a subject” thus particularly denotes “in vivo blood coagulation”.

In some embodiments, the invention provides the use of phosphorylated ACC in platelets from a subject for the diagnosis of blood coagulation in the subject.

In other embodiments, the invention provides the use of phosphorylated ACC in platelets from a subject for the monitoring of blood coagulation in the subject.

In a related aspect, the invention provides a method for determining blood coagulation comprising determining phosphorylated ACC in platelets from a subject.

In preferred embodiments, the invention provides a method for determining blood coagulation in a subject comprising determining phosphorylated ACC in platelets from the subject.

With the phrase “determining phosphorylated ACC” is meant herein an at least qualitative evaluation of the phosphorylation status of ACC in platelets from a subject, such as detecting whether ACC is phosphorylated or not. The term may but need not further encompass measuring the level of phosphorylated ACC.

In further embodiments, the invention provides a method for determining blood coagulation, comprising measuring the level of phosphorylated ACC in platelets from a subject.

In preferred further embodiments, the invention provides a method for determining blood coagulation in a subject, comprising measuring the level of phosphorylated ACC in platelets from the subject.

With the phrase “measuring phosphorylated ACC” is meant herein a quantitative evaluation of the phosphorylation status of ACC in platelets from a subject. Such quantification may without limitation denote relative quantification, e.g., determining the fraction of phosphorylated platelet ACC versus total platelet ACC in a sample, or determining the relative amount of phosphorylated ACC in a given amount of platelets or platelet protein or platelet protein fraction from a sample compared to a predetermined control amount of phosphorylated ACC; and/or may denote absolute quantification, e.g., determining the absolute amount (e.g., weight, moles, enzymatic units) of phosphorylated ACC in a given amount of platelets or platelet protein or platelet protein fraction from a sample.

In some embodiments, the invention provides a method for the diagnosis of blood coagulation in a subject comprising:
(i) determining phosphorylated ACC in platelets from the subject;
(ii) comparing phosphorylated ACC as determined in (i) with a reference value, said reference value representing a known status of blood coagulation;
(iii) finding a deviation or no deviation of phosphorylated ACC as determined in (i) from said reference value;
(iv) attributing said finding of deviation or no deviation to a particular diagnosis of blood coagulation in the subject.

Further embodiments of the invention relate to a system comprising:
a computer data repository that comprises a reference value, said reference value representing a known status of blood coagulation; and
a computer system programmed to access the data repository and to use information from the data repository in combination with information on phosphorylated ACC in platelets from a subject, to make a diagnosis of blood coagulation in the subject.

Related embodiments of the invention concern method for making a diagnosis of blood coagulation in a subject comprising:
receiving data representative of values of phosphorylated ACC in platelets from the subject;
accessing a data repository on a computer, said data repository comprising a reference value, said reference value representing a known status of blood coagulation; and
comparing the data representative of values of phosphorylated ACC in platelets from the subject with the reference value in the data repository on the computer, thereby making a diagnosis of blood coagulation in the subject. In certain embodiments, the determination of what action is to be taken, e.g., by a clinician, in view of said diagnosis is performed by a (the) computer.

In further embodiments of the methods for the diagnosis of blood coagulation in the subject as taught herein, the invention provides methods wherein in step (iv) higher phosphorylated ACC in platelets from the subject as compared to the reference value is attributed to increased blood coagulation compared to the blood coagulation status represented by the reference value.

In other embodiments, the invention provides a method for the monitoring of blood coagulation in a subject comprising:
(i) determining phosphorylated ACC in platelets from the subject from two or more successive time points;
(ii) comparing said phosphorylated ACC as determined in (i);
(iii) finding a deviation or no deviation of said phosphorylated ACC as compared in (ii);
(iv) attributing said finding of deviation or no deviation to a particular change in blood coagulation in the subject between the two or more successive time points.

In further embodiments of the methods for the monitoring of blood coagulation in the subject as taught herein, the invention provides methods wherein in step (iv) higher or lower phosphorylated ACC in platelets at a later one of the two or more successive time points than at an earlier one of the two or more successive time points is attributed to increased or decreased, respectively, blood coagulation at the later time point compared to the earlier time point.

As noted, blood coagulation as used herein is meant to encompass platelet activation as well as activation of the blood coagulation cascade.

In preferred embodiments of the uses or methods as taught herein, the blood coagulation is mediated by an agonist of a platelet thrombin receptor, preferably protease activated receptor (PAR), more preferably PAR1 and/or PAR4.

In further preferred embodiments of the uses or methods as taught herein, the blood coagulation is mediated by thrombin. Thrombin also represents an exemplary agonist of a platelet thrombin receptor.

Whereas the term “mediate” is known per se, it is particularly intended herein to mean “being effected by”, “acting through” or “involving”. As used herein, an agent that mediates blood coagulation refers to an agent that exhibits a direct or indirect causation, connection to or relation with or that is involved in blood coagulation, e.g. by inducing, promoting or stimulating blood coagulation, and may include, for example, but without limitation, agents that induce, promote or stimulate platelet activation and agents that are produced in the blood coagulation cascade.

In preferred embodiments of the uses or methods as taught herein, the blood coagulation thus comprises thrombin production.

In other preferred embodiments of the uses or methods as taught herein, the blood coagulation comprises the activation of a platelet thrombin receptor, preferably PAR, more preferably PAR1 and/or PAR4.

In yet other preferred embodiments of the uses or methods as taught herein, the blood coagulation comprises platelet activation, preferably platelet thrombin receptor-mediated platelet activation, more preferably thrombin-induced platelet activation.

In further preferred embodiments of the uses or methods as taught herein, the blood coagulation comprises platelet activation that results in platelet aggregation and/or platelet degranulation, preferably platelet thrombin receptor-mediated platelet activation that results in platelet aggregation and/or platelet degranulation, more preferably thrombin-induced platelet activation that results in platelet aggregation and/or platelet degranulation.

In particularly preferred embodiments of the uses or methods as taught herein, the blood coagulation comprises platelet aggregation, preferably platelet thrombin receptor-mediated platelet aggregation, more preferably thrombin-induced platelet aggregation.

Accordingly, phosphorylated ACC in platelets from a subject can be suitably used as a biomarker for thrombin production; or for activation of a platelet thrombin receptor, preferably PAR, more preferably PAR1 and/or PAR4; or for platelet activation, preferably platelet thrombin receptor-mediated platelet activation, more preferably thrombin-induced platelet activation; or for platelet aggregation and/or platelet degranulation; preferably in the subject.

Likewise, methods comprising same steps as the respective methods disclosed above aimed at determining of phosphorylated ACC in a subject could be suitably employed as methods for determining, diagnosing or monitoring thrombin production; or activation of a platelet thrombin receptor, preferably PAR, more preferably PAR1 and/or PAR4; or platelet activation, preferably platelet thrombin receptor-mediated platelet activation, more preferably thrombin-induced platelet activation; or platelet aggregation and/or platelet degranulation; preferably in the subject.

Accordingly, the present invention also provides the use of phosphorylated Acetyl-CoA carboxylase (ACC) in platelets from a subject as a biomarker: for thrombin production; or for activation of a platelet thrombin receptor, preferably PAR, more preferably PAR1 and/or PAR4; or for platelet activation, preferably platelet thrombin receptor-mediated platelet activation, more preferably thrombin-induced platelet activation; or for platelet aggregation and/or platelet degranulation.

In certain embodiments, the invention also provides the use of phosphorylated Acetyl-CoA carboxylase (ACC) in platelets from a subject as a biomarker for thrombin production; or for activation of a platelet thrombin receptor, preferably PAR, more preferably PAR1 and/or PAR4; or for platelet activation, preferably platelet thrombin receptor-mediated platelet activation, more preferably thrombin-induced platelet activation; or for platelet aggregation and/or platelet degranulation, in the subject.

In other embodiments, the invention also provides the use of phosphorylated ACC in platelets from a subject for the diagnosis or the monitoring of thrombin production; or activation of a platelet thrombin receptor, preferably PAR, more preferably PAR1 and/or PAR4; or platelet activation, preferably platelet thrombin receptor-mediated platelet activation, more preferably thrombin-induced platelet activation; or platelet aggregation and/or platelet degranulation, in the subject.

Further provided is a method for determining thrombin production; or activation of a platelet thrombin receptor, preferably PAR, more preferably PAR1 and/or PAR4; or platelet activation, preferably platelet thrombin receptor-mediated platelet activation, more preferably thrombin-induced platelet activation; or platelet aggregation and/or platelet degranulation, comprising determining phosphorylated ACC in platelets from a subject, preferably comprising measuring the level of phosphorylated ACC in the platelets from a subject.

In embodiments, the invention also provides a method for determining thrombin production; or activation of a platelet thrombin receptor, preferably PAR, more preferably PAR1 and/or PAR4; or platelet activation, preferably platelet thrombin receptor-mediated platelet activation, more preferably thrombin-induced platelet activation; or platelet aggregation and/or platelet degranulation, in a subject comprising determining phosphorylated ACC in platelets from the subject, preferably comprising measuring the level of phosphorylated ACC in the platelets from the subject.

In certain embodiments, the invention provides a method for the diagnosis of thrombin production; or activation of a platelet thrombin receptor, preferably PAR, more preferably PAR1 and/or PAR4; or platelet activation, preferably platelet thrombin receptor-mediated platelet activation, more preferably thrombin-induced platelet activation; or platelet aggregation and/or platelet degranulation, in a subject comprising:
(i) determining phosphorylated ACC in platelets from the subject;
(ii) comparing phosphorylated ACC as determined in (i) with a reference value, said reference value representing a known status of thrombin production; or activation of a platelet thrombin receptor, preferably PAR, more preferably PAR1 and/or PAR4; or platelet activation, preferably platelet thrombin receptor-mediated platelet activation, more preferably thrombin-induced platelet activation; or platelet aggregation and/or platelet degranulation;
(iii) finding a deviation or no deviation of phosphorylated ACC as determined in (i) from said reference value;
(iv) attributing said finding of deviation or no deviation to a particular diagnosis of thrombin production; or activation of a platelet thrombin receptor, preferably PAR, more preferably PAR1 and/or PAR4; or platelet activation, preferably platelet thrombin receptor-mediated platelet activation, more preferably thrombin-induced platelet activation; or platelet aggregation and/or platelet degranulation, in the subject.

Further embodiments of the invention relate to a system comprising:
a computer data repository that comprises a reference value, said reference value representing a known status of thrombin production; or of activation of a platelet thrombin receptor, preferably PAR, more preferably PAR1 and/or PAR4; or of platelet activation, preferably platelet thrombin receptor-mediated platelet activation, more preferably thrombin-induced platelet activation; or of platelet aggregation and/or platelet degranulation; and
a computer system programmed to access the data repository and to use information from the data repository in combination with information on phosphorylated ACC in platelets from a subject, to make a diagnosis of thrombin production; or of activation of a platelet thrombin receptor, preferably PAR, more preferably PAR1 and/or PAR4; or of platelet activation, preferably platelet thrombin receptor-mediated platelet activation, more preferably thrombin-induced platelet activation; or of platelet aggregation and/or platelet degranulation, in the subject.

Related embodiments of the invention concern method for making a diagnosis of thrombin production; or of activation of a platelet thrombin receptor, preferably PAR, more preferably PAR1 and/or PAR4; or of platelet activation, preferably platelet thrombin receptor-mediated platelet activation, more preferably thrombin-induced platelet activation; or of platelet aggregation and/or platelet degranulation, in a subject comprising:
receiving data representative of values of phosphorylated ACC in platelets from the subject;
accessing a data repository on a computer, said data repository comprising a reference value, said reference value representing a known status of thrombin production; or of activation of a platelet thrombin receptor, preferably PAR, more preferably PAR1 and/or PAR4; or of platelet activation, preferably platelet thrombin receptor-mediated platelet activation, more preferably thrombin-induced platelet activation; or of platelet aggregation and/or platelet degranulation; and
comparing the data representative of values of phosphorylated ACC in platelets from the subject with the reference value in the data repository on the computer, thereby making a diagnosis of thrombin production; or of activation of a platelet thrombin receptor, preferably PAR, more preferably PAR1 and/or PAR4; or of platelet activation, preferably platelet thrombin receptor-mediated platelet activation, more preferably thrombin-induced platelet activation; or of platelet aggregation and/or platelet degranulation, in the subject. In certain embodiments, the determination of what action is to be taken, e.g., by a clinician, in view of said diagnosis is performed by a (the) computer.

In further embodiments of the methods for the diagnosis of thrombin production; or activation of a platelet thrombin receptor; or platelet activation; or platelet aggregation and/or platelet degranulation as taught herein, the invention also provides a method wherein in step (iv) higher phosphorylated ACC in platelets from the subject as compared to the reference value is attributed to increased thrombin production; or activation of a platelet thrombin receptor, preferably PAR, more preferably PAR1 and/or PAR4; or platelet activation, preferably platelet thrombin receptor-mediated platelet activation, more preferably thrombin-induced platelet activation; or platelet aggregation and/or platelet degranulation compared to the status of thrombin production represented by the reference value.

In other embodiments, the invention also provides a method for the monitoring of thrombin production; or activation of a platelet thrombin receptor, preferably PAR, more preferably PAR1 and/or PAR4; or platelet activation, preferably platelet thrombin receptor-mediated platelet activation, more preferably thrombin-induced platelet activation; or platelet aggregation and/or platelet degranulation, in a subject comprising:
(i) determining phosphorylated ACC in platelets from the subject from two or more successive time points;
(ii) comparing said phosphorylated ACC as determined in (i);
(iii) finding a deviation or no deviation of said phosphorylated ACC as compared in (ii);
(iv) attributing said finding of deviation or no deviation to a particular change in thrombin production; or activation of a platelet thrombin receptor, preferably PAR, more preferably PAR1 and/or PAR4; or platelet activation, preferably platelet thrombin receptor-mediated platelet activation, more preferably thrombin-induced platelet activation; or platelet aggregation and/or platelet degranulation in the subject between the two or more successive time points.

In further embodiments of the methods for the monitoring of thrombin production; or activation of a platelet thrombin receptor; or platelet activation; or platelet aggregation and/or platelet degranulation as taught herein, the invention provides methods wherein in step (iv) higher or lower phosphorylated ACC in platelets at a later one of the two or more successive time points than at an earlier one of the two or more successive time points is attributed to increased or decreased, respectively, thrombin production; or activation of a platelet thrombin receptor, preferably PAR, more preferably PAR1 and/or PAR4; or platelet activation, preferably platelet thrombin receptor-mediated platelet activation, more preferably thrombin-induced platelet activation; or platelet aggregation and/or platelet degranulation at the later time point compared to the earlier time point.

Such activities may be studied or evaluated in contexts other than bleeding, such as for example but without limitation in sepsis or diabetes. Indeed, in diabetic patients, platelet dysfunction is commonly observed.

Accordingly, the present invention also provides the use of phosphorylated Acetyl-CoA carboxylase (ACC) in platelets from a subject as a biomarker for platelet aggregation, preferably platelet thrombin receptor-mediated platelet aggregation, more preferably thrombin-induced platelet aggregation.

In certain embodiments, the invention also provides the use of phosphorylated Acetyl-CoA carboxylase (ACC) in platelets from a subject as a biomarker for platelet aggregation, preferably platelet thrombin receptor-mediated platelet aggregation, more preferably thrombin-induced platelet aggregation, in the subject.

In other embodiments, the invention also provides the use of phosphorylated ACC in platelets from a subject for the diagnosis or the monitoring of platelet aggregation, preferably platelet thrombin receptor-mediated platelet aggregation, more preferably thrombin-induced platelet aggregation, in the subject.

Further provided is a method for determining platelet aggregation, preferably platelet thrombin receptor-mediated platelet aggregation, more preferably thrombin-induced platelet aggregation, comprising determining phosphorylated ACC in platelets from a subject, preferably comprising measuring the level of phosphorylated ACC in the platelets from a subject.

In embodiments, the invention also provides a method for determining platelet aggregation, preferably platelet thrombin receptor-mediated platelet aggregation, more preferably thrombin-induced platelet aggregation, in a subject comprising determining phosphorylated ACC in platelets from the subject, preferably comprising measuring the level of phosphorylated ACC in the platelets from the subject.

In certain embodiments, the invention provides a method for the diagnosis of platelet aggregation, preferably platelet thrombin receptor-mediated platelet aggregation, more preferably thrombin-induced platelet aggregation, in a subject comprising:
(i) determining phosphorylated ACC in platelets from the subject;
(ii) comparing phosphorylated ACC as determined in (i) with a reference value, said reference value representing a known status of platelet aggregation, preferably platelet thrombin receptor-mediated platelet aggregation, more preferably thrombin-induced platelet aggregation;
(iii) finding a deviation or no deviation of phosphorylated ACC as determined in (i) from said reference value;
(iv) attributing said finding of deviation or no deviation to a particular diagnosis of platelet aggregation, preferably platelet thrombin receptor-mediated platelet aggregation, more preferably thrombin-induced platelet aggregation, in the subject.

In further embodiments of the methods for the diagnosis of platelet aggregation as taught herein, the invention also provides a method wherein in step (iv) higher phosphorylated ACC in platelets from the subject as compared to the reference value is attributed to increased platelet aggregation, preferably platelet thrombin receptor-mediated platelet aggregation, more preferably thrombin-induced platelet aggregation, compared to the status of platelet aggregation, preferably platelet thrombin receptor-mediated platelet aggregation, more preferably thrombin-induced platelet aggregation represented by the reference value.

In other embodiments, the invention also provides a method for the monitoring of platelet aggregation, preferably platelet thrombin receptor-mediated platelet aggregation, more preferably thrombin-induced platelet aggregation, in a subject comprising:
(i) determining phosphorylated ACC in platelets from the subject from two or more successive time points;
(ii) comparing said phosphorylated ACC as determined in (i);
(iii) finding a deviation or no deviation of said phosphorylated ACC as compared in (ii);
(iv) attributing said finding of deviation or no deviation to a particular change in platelet aggregation, preferably platelet thrombin receptor-mediated platelet aggregation, more preferably thrombin-induced platelet aggregation, in the subject between the two or more successive time points.

In further embodiments of the methods for the monitoring of blood coagulation in the subject as taught herein, the invention provides methods wherein in step (iv) higher or lower phosphorylated ACC in platelets at a later one of the two or more successive time points than at an earlier one of the two or more successive time points is attributed to increased or decreased, respectively, platelet aggregation, preferably platelet thrombin receptor-mediated platelet aggregation, more preferably thrombin-induced platelet aggregation at the later time point compared to the earlier time point.

In certain embodiments of the uses or methods as taught herein, the subject has been administered an anti-coagulant or a pro-coagulant. Hereby, the present uses or methods may advantageously allow to test the effectiveness of such anti-coagulants or pro-coagulants in the subject, or test the responsiveness of the subject to such anti-coagulants or pro-coagulants.

In further embodiments of the uses or methods as taught herein, the subject has been administered an anti-coagulant selected from the group comprising or consisting of an inhibitor of thrombin production, an inhibitor of thrombin activity (e.g. a direct thrombin inhibitor), an inhibitor of binding of thrombin to a platelet thrombin receptor, a platelet thrombin receptor antagonist, inhibitor of platelet activation, preferably platelet thrombin receptor-mediated platelet activation, more preferably thrombin-induced platelet activation or a pro-coagulant selected from the group comprising an activator of thrombin production or thrombin activity, thrombin, a platelet thrombin receptor agonist.

The uses or methods discloses herein may be particularly useful for evaluating blood coagulation processes in patients which suffer from a coagulation disorder, e.g., a disorder causing inadequate or excessive blood coagulation.

Hence, in other embodiments of the uses or methods as taught herein, the subject is a patient with a blood coagulation disorder.

The invention further relates to uses and methods for screening for anti-coagulants or pro-coagulants.

In an aspect, the invention thus provides the use of phosphorylated ACC in platelets from a subject for screening of one or more test agents for a candidate anti-coagulant or pro-coagulant, wherein the subject has been administered the test agent.

In a related aspect, the invention provides a method for screening one or more test agents for a candidate anti-coagulant or pro-coagulant, comprising determining whether phosphorylated ACC is modulated in platelets from a subject, wherein the subject has been administered the test agent.

In preferred embodiments, the invention provides a method for screening one or more test agents for a candidate anti-coagulant or pro-coagulant comprising:

(i) contacting a test agent with platelets;

(ii) determining whether said test agent can modulate phosphorylated ACC in said platelets.

The terms “modulate” or “modulated” or derivatives thereof are used in their broadest sense herein, and may particularly denote changing or modifying in any direction and to any extent a process, property, function or variable, etc. that is said to be so modulated. For example, in respective contexts, modulating may carry the meanings of stimulating, inhibiting, preventing or providing, etc. The modulation may reflect qualitative and/or quantitative change(s), and specifically encompasses both: increase (e.g., activation or stimulation) or decrease (e.g., inhibition), of that which is being modulated.

In preferred embodiments of the uses or methods for screening the one or more test agents for a candidate anti-coagulant or pro-coagulant as taught herein, the anti-coagulant is selected from the group comprising or consisting of an inhibitor of thrombin production, an inhibitor of thrombin activity (e.g. a direct thrombin inhibitor), an inhibitor of binding of thrombin to a platelet thrombin receptor, a platelet thrombin receptor antagonist, inhibitor of platelet activation, preferably platelet thrombin receptor-mediated platelet activation, more preferably thrombin-induced platelet activation or a pro-coagulant selected from the group comprising an activator of thrombin production or thrombin activity, thrombin, a platelet thrombin receptor agonist. Such inhibitors, antagonists, activators or agonists may be know to have such activity (e.g., in other test system(s)) or may be otherwise suspected to have such activity (e.g., based on structural or functional predictions).

These and further aspects and preferred embodiments of the invention are described in the following sections and in the appended claims. The subject matter of appended claims is hereby specifically incorporated in this specification.

### BRIEF DESCRIPTION OF FIGURES

FIG. 1 Thrombin induces AMPK activation in vitro via the stimulation of CaMKKβ. (A) Western blot analysis of the expression of the α1 subunit and the α2 subunit of AMPK in extracts from mesenchymal stem cells (MSC), liver, heart and human platelets. (B) Activation of the α1 subunit and the α2 subunit of AMPK in human platelets following treatment with 0.1 U/ml thrombin. (C) Western blot analysis of CaMKKβ expression in human platelets, brain and vascular smooth muscle cells (VSMC). (D) Western blot analysis of phosphorylated(Ser79) ACC in human platelets following treatment with 0.1 U/ml thrombin alone or with a combination of thrombin and 10 μM STO-069.

FIG. 2 STO-609 prevents platelet aggregation and platelet degranulation in vitro. (A,B) Aggregation of human platelets following treatment with 0.025 U/ml thrombin (A) or 20 μM ADP (B) in the absence or presence of 10 μM STO-609. (C) Flow cytometric analysis of the activation of integrin αIIbβ3 on the surface of human platelets following treatment with 0.025 U/ml thrombin in the absence or presence of 10 μM STO-609. (D) Flow cytometric analysis of the secretion of α-granules by human platelets following treatment with 0.025 U/ml thrombin in the absence or presence of 10 μM STO-609.

FIG. 3 STO-609 prevents MLC and VASP phosphorylation in vitro. (A, B) Western blot analysis of MLC phosphorylation (Ser19) (A) and VASP phosphorylation (Thr278) (B) in human platelets following treatment with 0.1 U/ml thrombin in the absence or presence of STO-609.

FIG. 4 Platelet aggregation and MLC phosphorylation is decreased in AMPK α1-knockout mice. (A) Western blot analysis of the expression of AMPK, and its α1 and α2 subunits in extracts from human platelets, platelets from wild-type (WT) mice, platelets from α1-AMPK knockout (KO) mice, mesenchymal stem cells (MSC) and heart. (B) Aggregation of platelets from wild-type (WT) or α1-AMPK knockout (KO) mice following treatment with 0.1 U/ml thrombin.

FIG. 5 Phosphorylation of ACC in platelets from a subject reflects coagulation cascade activation in said subject. (A-F) Western blot analysis of phosphorylated(Ser79) ACC in human platelets from 6 patients who underwent cardiac surgery with a cardio-pulmonary bypass procedure (CPB). The human platelets were isolated from blood samples drawn during CPB and 4 hours after surgery. (G) Graph showing individual ACC phosphorylation in human platelets from the 6 patients in the post-operative state compared to the CPB state. (H) Graph showing mean ACC phosphorylation in human platelets from the 6 patients in the post-operative state compared to the CPB state. (I) Graph showing correlation between post-operative bleeding severity characterized by chest tube drainage and increased ACC phosphorylation in the post-operative state compared to the CPB state.

FIG. 6 Phosphorylation of ACC in platelets from a subject reflects coagulation cascade activation and platelet activation in said subject. Blood samples were drawn from patients (n=29) who underwent cardiac surgery with a cardio-pulmonary bypass procedure. (A) Graph showing mean ACC phosphorylation in platelets in the post-operative state compared to the CPB state. Platelets were isolated from blood samples drawn during surgery (CPB) and 4 hours after surgery (post-op). Platelet lysates were subjected to Western blot analysis with anti-phospho Ser 79 ACC antibodies and phosphorylated ACC was quantified by densitometry normalized to GAPDH expression and to a calibrator sample. The data are expressed as means±SEM (n=29, p=0.057). (B) Automated thrombogram showing the amount of thrombin generated from a patients plasma sample (before surgery (pre-op), during surgery (CPB) and after surgery (post-op)) plotted against time as calculated using a fluorogenic thrombin generation assay. (C) Graph showing ETP values in plasma samples in the post-operative state compared to the CPB state as measured using a fluorogenic thrombin generation assay. (D) Graph showing increase in ACC phosphorylation (post-operation versus CBP) in patients with a poor recovery of ETP after CBP (ETP<20%; post-operation versus basal) or patients preserving an intact ability to generate thrombin after CBP (ETP>80%; post-operation versus basal). Data are represented as means±SEM (*p<0.057). (E) Graph showing correlation between post-operative bleeding severity characterized by chest tube drainage and increased ACC phosphorylation in the post-operative state compared to the CPB state in patients with a positive TRAP test (≧830 A.U.) and without transfusion of platelets during CPB.

FIG. 7 ACC phosphorylation in human platelets is specifically induced by thrombin. Western blot analysis of phosphorylated(Ser79) ACC in isolated washed human platelets following treatment for 1 minute with increasing doses of thrombin (0.01, 0.03, 0.1, 0.3 and 1 U/ml), the thromboxane A2 (TXA2) receptor agonist U46619 (0.1, 0.3, 1, 3 and 10 μM), collagen (0.5, 1, 2.5, 5 and 10 μg/ml) or ADP (2.5, 5, 10, 25 nd 50 μM). Gelsolin expression was used as a loading control.

FIG. 8 The CAMKKβ/AMPK pathway does not affect TXA2 production or Ca²⁺ release. (A) Fluo-3-loaded platelets were pre-incubated with 10 μM STO-609 for 2 min at 37° C. in the presence of 1 mM extracellular Ca²⁺ and 0.1 U/ml thrombin was then added to trigger the increase of intracellular Ca²⁺. (B) TXB2 levels were assayed in supernatants of platelets that were pre-incubated with 10 μM STO-609 or DMSO prior to treatment with 0.1 U/ml thrombin. Platelets activated by thrombin in the presence of 1 mM acetylsalicylic acid (ASA) are negative controls. Similar results were obtained in 3 separate experiments. The data are expressed as mean±SEM (***P<0.001 compared to corresponding control values).

FIG. 9 STO-609 prevents cofilin phosphorylation in vitro. Washed platelets were incubated with thrombin 0.1 U/ml in the presence (triangle) or absence (rectangular) of 10 μM STO-609, and lysates were harvested for Western blotting and densitometry. A representative blot of cofilin phosphorylation state is shown in the upper panel. Cofilin expression was normalized to gelsolin expression. Quantitative results were plotted in a histogram (lower panel). The data are mean±SEM (n=5, P<0.05 compared with corresponding control values).

FIG. 10 ACC, MLC, VASP and cofilin phosphorylation is decreased in AMPK α1-knockout mice. Purified murine platelets isolated from WT (filled bars) or AMPKα1 KO (unfilled bars) mice were stimulated by 0.1 U/ml thrombin for the indicated times. Platelet lysates were analyzed by immunoblotting with anti-phospho Ser 79 ACC (A), anti-phospho Ser 19 MLC (B), anti-phospho Thr 178 VASP (C) or anti-phospho Ser 3 cofilin (D) antibodies and quantified by densitometry. ACC, MLC, VASP and cofilin expression was normalized to GAPDH expression. The data are expressed as mean±SEM (n=5, *P<0.05, **P<0.01 and ***P<0.001 compared to thrombin values in KO; #P<0.05, ##P<0.01 and ## #P<0.001 compared to control value in WT; $P<0.05 compared to control value in KO).

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The term also encompasses “consisting of” and “consisting essentially of”.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the term “one or more”, such as one or more members of a group of members, is clear per se, by means of further exemplification, the term encompasses inter alia a reference to any one of said members, or to any two or more of said members, such as, e.g., any ≧3, ≧4, ≧5, ≧6 or ≧7 etc. of said members, and up to all said members.

All documents cited in the present specification are hereby incorporated by reference in their entirety. In particular, the teachings or sections of such documents herein specifically referred to are incorporated by reference.

The discussion of the background to the invention herein is included to explain the context of the present invention. This is not to be taken as an admission that any of the material referred to was published, known, or part of the common general knowledge in any country as of the priority date of any of the claims.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention. When specific terms are defined in connection with a particular aspect or embodiment, such connotation is meant to apply throughout this specification, i.e., also in the context of other aspects or embodiments, unless otherwise defined.

As noted, the inventors surprisingly realised that phosphorylated Acetyl-CoA carboxylase (ACC) in platelets from a subject can be used as a biomarker for blood coagulation, preferably a biomarker for blood coagulation in the subject.

In a certain way, the methods and uses disclosed herein may be denoted as in vitro methods, for example since they comprise determination of platelet ACC phosphorylation in a sample removed from a subject. The term “in vitro” generally denotes outside, or external to, a body, e.g., an animal or human body. The term “ex vivo” typically refers to tissues or cells removed from a body, e.g., an animal or human body, and maintained or propagated outside the body, e.g., in a culture vessel. The term “in vitro” as used herein should be understood to include “ex vivo”.

The terms “biomarker” or “marker” are widespread in the art and may broadly denote a substance whose qualitative and/or quantitative evaluation in a subject is predictive or informative with respect to one or more aspects of the subject's phenotype and/or genotype. As used herein, the terms “biomarker” or “marker” relate to a substance, in particular phosphorylated platelet Acetyl-CoA carboxylase, which is used as an indicator for blood coagulation.

The term “Acetyl-CoA carboxylase” or “ACC” refers in general to a biotin-dependent enzyme that catalyzes the irreversible carboxylation of acetyl-CoA to produce malonyl-CoA and hence, ACC is mainly involved in regulating the metabolism of fatty acids. ACC is a multi-subunit enzyme in most prokaryotes and in the chloroplasts of most plants and algae, whereas it is a large, multi-domain enzyme in the endoplasmic reticulum of most eukaryotes. In mammals two forms of ACC are expressed: ACC-alpha, ACC1, ACCA or ACACA and ACC-beta, ACC2, ACCB or ACACB and the term encompasses both forms. Exemplary human ACC1 protein sequence may be as annotated under NCBI Genbank accession number NP_{—}942131.1 (isoform 1) (sequence version 1), NP_{—}942133.1 (isoform 2) (sequence version 1), NP_{—}942136.1 (isoform 2) (sequence version 1), NP_{—}942134.1 (isoform 3) (sequence version 1) or NP_{—}942135.1 (isoform 4) (sequence version 1) and exemplary human ACC2 protein sequence may be as annotated under NCBI Genbank accession number NP_{—}001084.3 (sequence version 3). Exemplary human ACC1 mRNA (cDNA) sequence may be as annotated under NCBI Genbank accession number NM_{—}198834.1 (transcript variant 1) (sequence version 1), NM_{—}198839.1 (transcript variant 2) (sequence version 1), NM_{—}198836.1 (transcript variant 3) (sequence version 1), NM_{—}198837.1 (transcript variant 4) (sequence version 1) or NM_{—}198838.1 (transcript variant 5) or (sequence version 1) and exemplary human ACC2 mRNA (cDNA) sequence may be as annotated under NCBI Genbank accession number NM_{—}001093.3 (sequence version 3).

As used herein, the term “Acetyl-CoA carboxylase” or “ACC” particularly refers to platelet Acetyl-CoA carboxylase. For example and without limitation, the term may refer to either one or more or all forms of ACC present in the platelets of a subject being tested. Hence, for example and without limitation, for human subjects or samples the term may refer to either one or both of ACC-alpha and ACC-beta.

The term “phosphorylated ACC” refers herein to ACC containing one or more phosphate groups, and particularly refers to ACC which is phosphorylated at least at its Ser79 residue (i.e., ACC which comprises a phosphate group added at least at its Ser79 residue). The phosphorylated ACC may but need not contain phosphate group(s) at residue(s) other than Ser79.

As used herein, the term “determining” is synonymous for “assessing” and includes “detecting” as well as “measuring”. The phrase “determining phosphorylated ACC” can be understood as “determining a value for phosphorylated ACC”.

“Value” as used herein in the context of “value for phosphorylated ACC” or “reference value” (especially, “reference value for phosphorylated ACC”) may refer to a qualitative phosphorylation status or condition of ACC, such as phosphorylated or not phosphorylated, as well as to the level, i.e., amount or quantity, of phosphorylated ACC, encompassing both the absolute level of phosphorylated ACC and the relative or normalised level of phosphorylated ACC.

The distinction between “absolute” and “relative” quantification is well appreciated by a skilled person. For example, absolute level of phosphorylated ACC may refer to the amount or quantity of phosphorylated ACC that correlates with the detected signal representing phosphorylated ACC. Absolute level of phosphorylated ACC may be expressed without limitation as absolute intensity of the detected signal representing phosphorylated ACC, or where possible converted to the weight, molar amount, enzymatic units (or similar quantifier) of phosphorylated ACC, measured typically in a given amount of platelets or platelet protein or platelet protein fraction from a sample. For example, “relative or normalised level of phosphorylated ACC” may be understood as the absolute level of phosphorylated ACC divided by or normalised to the total level of ACC, i.e., the sum of the level of phosphorylated ACC and the level of non-phosphorylated ACC. Otherwise, relative quantification may involve determining the relative amount of phosphorylated ACC in a given amount of platelets or platelet protein or platelet protein fraction from a sample compared to a predetermined control amount of phosphorylated ACC.

Techniques for determining or assessing a phosphorylated protein are well known in the art and include for example, but without limitation, Western blot analysis, Enzyme-Linked Immunoabsorbent Assay (ELISA), cell-based ELISA, flow cytometry, immunocytochemistry (ICC) or immunohistochemistry (IHC), mass spectrometry and multi-analyte profiling.

Western blot analysis is the most common method used for assessing the phosphorylation status of a protein. Following separation of the sample with SDS-PAGE and subsequent transfer to a membrane, a phospho-specific antibody is used to identify the target protein. Because the measured levels of the phosphorylated protein may change through gel loading errors, one may utilise an antibody that detects the total level of the target protein (regardless of its phosphorylation status) to determine the phosphorylated fraction relative to the total fraction and to serve as an internal loading control. Radioisotope, chemiluminescent and colorimetric detection methods are common, and molecular weight markers are generally used to provide information about protein mass.

The ELISA has become a powerful technique for determining protein phosphorylation status. ELISAs are in general more quantitative than Western blot analysis. The format for this advantageously microplate-based assay typically utilises a capture antibody specific for the target protein, independent of its phosphorylation status. The target protein is then bound to the antibody-coated plate. A detection antibody that specifically recognises the phosphorylated target protein is then added. These assays are typically designed using colorimetric or fluorometric detection. The intensity of the resulting signal is directly proportional to the concentration of phosphorylated target protein present in the sample and by utilising a calibrated standard results are easily quantifiable. The ELISA may be done manually or automated.

Recently, several immunoassays, known as cell-based ELISAs, enabling the determination of protein phosphorylation in the context of a whole cell have been developed. The cells are fixed, and blocked in the same well. Phospho-specific antibodies are used to assess phosphorylation status using fluorometric or colorimetric detection systems. These assays bypass the need for the creation of cell lysates and are therefore more amenable to high throughput analyses.

For determination of phosphorylated proteins by flow cytometry and ICC/IHC cells are usually fixed with formaldehyde or paraformaldehyde to cross-link the phosphorylated proteins and stabilize them for analysis. The fixed cells must then be permeabilised to allow for entry of phospho-specific antibodies into the cells.

Flow cytometry uses a laser to excite the fluorochrome used for antibody detection. Flow cytometry is advantageous because it allows for rapid, quantitative, single cell analysis. Proteins can be detected in a specific cell type within a heterogeneous population via cell surface marker phenotyping without the need to physically separate the cells. Filter sets and fluorochromes with non-overlapping spectra are then carefully chosen to assess multiple proteins in the same cell.

ICC generally refers to protein detection by microscopy in cultured cells, while IHC refers to protein detection in intact tissue sections. Like flow cytometry, these techniques allow for the assessment of multiple proteins within a cell or tissue provided that adequate attention is given to avoid overlapping fluorescence spectra or colour. Both fluorescent and colorimetric detection techniques are commonly used.

Mass spectrometry (MS) techniques may also be useful tools for determining phosphorylated proteins. However, there are several inherent difficulties for the analysis of phospho-proteins since signals from phosphopeptides are generally weaker and it can be difficult to observe the signals from low-abundance phosphorylated target proteins in the high-background of abundant non-phosphorylated proteins. To overcome these drawbacks, several enrichment strategies for phospho-protein analysis by MS have been developed including immobilized metal affinity chromatography (IMAC) (Brill, L. M. et al. 2004. Anal. Chem. 76:2763), phospho-specific antibody enrichment (Steen, H. et al. 2002. J. Biol. Chem. 277:1031), chemical-modification-based methods such as beta-elimination of phospho-serine and -threonine (Zhou, H. et al. 2001. Nat. Biotechnol. 19:375), and replacement of the phosphate group with biotinylated moieties (Oda, Y. et al. 2001. Nat. Biotechnol. 19:379).

Multiple analyte profiling is a mass spectrometric technique such as collision-induced dissociation (CID) or electron transfer dissociation (ETD) that determines protein phosphorylation of multiple analytes simultaneously. It involves the use of phospho-specific antibodies and include microplate-based, bead-based, or membrane-based detection formats. The obvious benefit of these assays is that throughput capability is greatly enhanced by bypassing the need for running multiple individual Western blots or traditional ELISA-based assays. These techniques are also known for providing more data while requiring very little sample volume. In trade, protein profiling assays are typically recognized as being less sensitive than their more conventional counterparts due to potential antibody cross-reactivity.

The ALPHAScreen™ technology developed by Perkin Elmer (see, e.g., “A Practical Guide to Working with AlphaScreen™”, PerkinElmer LAS literature, 007011_{—}01. Boston: PerkinElmer, 2004), based on methodology originally developed under the name LOCI (Luminescent Oxygen Channeling Immunoassay) by Dade Behring, Inc. of Germany (see, e.g., Ullman et al. Proc Natl Acad Sci USA 1994, 91: 5426-5430; and Ullman et al. Clin Chem 1996, 42: 1518-1526), is a bead-based, non-radioactive Amplified Luminescent Proximity Homogeneous Assay. In this assay, a donor and an acceptor pair of microbeads are brought into proximity by a biomolecular interaction of binding partners immobilized to these beads. Excitation of the assay mixture with a high-intensity laser at 680 nm induces the formation of singlet oxygen at the surface of the donor bead (following conversion of ambient oxygen to a more excited state by a photosensitizer present in the donor bead). The singlet oxygen molecules can diffuse up to 200 nm. If an acceptor bead is in proximity, the singlet oxygen can react with a thioxene derivative present in this bead, generating chemiluminescence at 370 nm that further activates the fluorophores contained in the same bead. The fluorophores subsequently emit light at 520-620 nm. The donor bead generates about 60,000 singlet oxygen molecules, resulting in an amplified signal. To assess the phosphorylation status of a protein using this ALPHAScreen™ technology, suitable binding partners may be a phospho-specific anti-target antibody, which specifically recognizes the target protein when it is phosphorylated, preferably an anti-phospho-ACC antibody, bound on one bead and a total anti-target antibody, which recognizes the target protein irrespective its phosphorylation status, preferably an anti-ACC antibody, bound on the other bead. Such assay kit, preferably an assay kit for detecting phosphorylated ACC, allows to get a quantitative value for the phosphorylation status of a target protein, preferably ACC.

Hence, in certain embodiments, phosphorylated ACC may be preferably detected using such ALPHAScreen™ based technology.

Hence, in certain embodiments, phosphorylated ACC may be detected by an assay comprising:
first (donor) microbeads and second (acceptor) microbeads,
wherein the donor microbeads are configured to release singlet oxygen when illuminated by a high-intensity laser, preferably at 680 nm (for example, the donor microbeads may comprise photosensitizer capable of converting ambient oxygen to an excited state),
wherein the acceptor microbeads comprise a thioxene derivative capable of reacting with said singlet oxygen, whereby said reaction generates chemiluminescence, preferably at 370 nm,
wherein the acceptor microbeads further comprise fluorophore configured to be activated by the light output of said chemiluminescence and to emit light, preferably at 520-620 nm, when so activated (by means of explanation, the light output of the fluorophore thus constitutes the readout of the assay),
wherein either one of the donor microbeads or acceptor microbeads comprise an antibody capable of specifically binding to phosphorylated ACC, particularly to ACC phosphorylated at least on Ser79 (i.e., a phospho-specific anti-ACC antibody), and
wherein the other one of the donor microbeads or acceptor microbeads comprise an antibody capable of binding to ACC irrespective of its phosphorylation status, particularly preferably irrespective of its phosphorylation status on Ser79.

Further, in an aspect, the invention provides a kit comprising the donor microbeads and acceptor microbeads as defined in the previous paragraph. Such kit may optionally also comprise suitable reagents (e.g., buffers) for performing the necessary reactions (e.g., reagents conducive to binding of the donor and acceptor microbeads to the phosphorylated ACC target), suitable controls (e.g., known quantities of phosphorylated ACC or non-phosphorylated ACC), reagents necessary for preparing platelet extract in which phosphorylated ACC can be measured, instructions for use, etc. Any of the components of the kit may be provided in separate vials or in combinations where applicable.

The majority of the above described techniques rely on the use of phospho-specific antibodies, i.e., antibodies that specifically recognise a target protein when it is phosphorylated. Phospho-specific antibodies may be obtained by immunising animals, e.g., rabbits, with phosphorylated proteins or synthetic phospho-peptides representing the amino acid sequence surrounding the phosphorylation site seen in the target protein. These peptides should be sufficient to form an epitope. These peptides may for example comprise from 6 to 20, preferably from 6 to 15, more preferably from 6 to 10 residues of amino acids. Preferably, from the generated antibodies those with high affinity and specificity for the antigen comprising the phosphorylated target protein are selected. Nowadays, phospho-specific antibodies are commercially available for most target proteins.

Techniques for determining or assessing phosphorylated ACC may typically rely on a phospho-ACC (Ser79) antibody that specifically recognizes ACC when phosphorylated at Ser79, such as for example the commercial available phospho-ACC (Ser79) antibodies such as Phospho-Acetyl-CoA Carboxylase (Ser79) Antibody (#3661, Cell Signaling Technology®), Acetyl Coenzyme A Carboxylase (phospho S79) antibody (ab31931, Abcam) and Anti-phospho-Acetyl CoA Carboxylase (Ser79) (07-303, Millipore). Suitable techniques for determining phosphorylated ACC may include PathScan® Phospho-Acetyl-CoA Carboxylase (Ser79) Sandwich ELISA kit (#7986, Cell Signaling Technology®).

Phosphorylated ACC is determined herein in “platelets from a subject”, which refers herein to a biological sample of a subject comprising platelets and may include, for example, but without limitation, undiluted and/or diluted whole blood, undiluted and/or diluted blood plasma, fraction(s) of fractionated whole blood such as the platelet rich plasma (PRP) fraction or isolated platelets.

The plasma-rich fraction from whole blood can be generally obtained by centrifugation of the blood at between 100 g and 400 g for between 5 and 20 min, such as, for example, by centrifugation at 150 g for 10 min or at 330 g for 15 min. A well-known method for purification of the platelet-rich fraction that allows for isolation of platelets is the OptiPrep™ method, which is based on density gradient centrifugation. There exist also methods that allow for separation of platelets from whole blood without centrifugation such as the method disclosed in WO200210771 using paramagnetic microparticles coated with antibodies that specifically bind to platelets.

The terms “subject” specifically refer to humans, but may also encompass reference to non-human animals, preferably warm-blooded animals, more preferably non-human mammals, such as, e.g., non-human primates, rodents, canines, felines, equines, ovines, porcines, and the like.

As noted in some preferred embodiments, the subject has been administered an anti-coagulant or a pro-coagulant. Suitable but non-limiting anti-coagulants include, for example, Bivaluridin-Angiox®, which is commercially available and currently used and oral anti-IIa (Dabigatran-Pradaxar®) and anti-Xa (Rivaroxaban-Xarelto®), which are expected to be marketed in the near future.

In some other preferred embodiments, the subject is a patient with a blood coagulation disorder.

The term “patient” refers herein a subject that is, or is suspected to be afflicted with a blood coagulation disorder. With the term “blood coagulation disorder” is meant herein any disorder that affects blood coagulation in a subject and encompasses blood clotting disorders as well as bleeding disorders. The term “blood clotting disorder” refers to a condition or disease characterised by an increased tendency, often repeated and over an extended period of time, for excessive clotting or thrombosis. Examples of clotting disorders include inter alia Factor V Leiden, protein C deficiency, protein S deficiency, anti-thrombin deficiency, prothrombin 20210A mutations and thrombotic disorders such as, for example, but without limitation, acute thrombotic stroke, venous thrombosis, myocardial infarction, unstable angina, abrupt closure following angioplasty or stent placement and thrombosis as a result of peripheral vascular surgery. The term “bleeding disorder” refers to a condition or disease characterized in poor blood clotting and continuous bleeding. Examples of bleeding disorders include inter alia Von Willebrand's disease, hemophilia, including hemophilia A and hemophilia B, Glanzmann's thrombasthenia, Bernard-Soulier syndrome, thrombocytopenia, vitamin K deficiency, amyloidosis, platelet dysfunction, systemic lupus erythematosus and immune thrombocytopenic purpura.

The terms “diagnosis” and “monitoring” are commonplace and well-understood in medical practice.

By means of further explanation and without limitation the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). As used herein, “diagnosis” of blood coagulation in a subject may particularly mean the determination of the presence (occurrence) or absence of blood coagulation in the subject.

The term “monitoring” generally refers to the follow-up of a disease or a condition in a subject for any changes which may occur over time. As used herein, “monitoring” blood coagulation in a subject thus particularly refers to the follow-up of the blood coagulation status in the subject for any changes in blood coagulation which may occur over time.

In the methods as taught herein, phosphorylated ACC determined in platelets from a subject may be compared with a reference value for said phosphorylated ACC representing a given status or condition of blood coagulation, such as, e.g., a known status of blood coagulation.

By means of example, but without limitation, a suitable reference value for phosphorylated ACC may—depending on the preference and intention of the experimenter—be the value of phosphorylated ACC in platelets from a subject is known to have no blood coagulation at the time of sampling, or is known to have blood coagulation at the time of sampling, or a subject who has been administered an anti-coagulant or a pro-coagulant.

The present methods may employ reference values for phosphorylated ACC that may be established according to known procedures previously employed for other biomarkers. Such reference values may be established either within (i.e., constituting a step of) or external to (i.e., not constituting a step of) the methods of the present invention as defined herein. Accordingly, any one of the methods taught herein may comprise a step of establishing a reference value for phosphorylated ACC. Such reference value may conveniently represent either (a) diagnosis of the absence of blood coagulation, or (b) diagnosis of the occurrence of blood coagulation, or (c) diagnosis of the occurrence of a certain known extent of blood coagulation.

Hence, also provided is a method for establishing a reference value for phosphorylated ACC, said reference value representing:

(a) diagnosis of the absence of blood coagulation, or

(b) diagnosis of the occurrence of blood coagulation, or

(c) diagnosis of the occurrence of a certain known extent of blood coagulation;

comprising:

(i) determining phosphorylated ACC in:
(i a) platelets from one or more samples from one or more subjects not having blood coagulation at the time of sampling, or
(i b) platelets from one or more samples from one or more subjects having blood coagulation at the time of sampling, or
(i c) platelets from one or more samples from one or more subjects having a certain known extent of blood coagulation at the time of sampling;
   and

(ii) storing the determined value of phosphorylated ACC:
(ii a) as determined in (i a) as the reference value representing the diagnosis of the absence of blood coagulation, or
(ii b) as determined in (i b) as the reference value representing the diagnosis of the occurrence of blood coagulation, or
(ii c) as determined in (i c) as the reference value representing the diagnosis of the occurrence of a certain known extent of blood coagulation.

In other methods as taught herein, phosphorylated ACC determined in platelets from a subject from two or more successive time points may be compared with each other. Typically, an action or event took place between said two or more time points, such as for example, the administration of an anti-coagulant or pro-coagulant, whereby the outcome of the method may be advantageously informative as to the effect of the administered anti-coagulant or pro-coagulant.

Such comparisons may generally include any means to determine the presence or absence of at least one difference and optionally of the magnitude of such difference between values being compared. A comparison may include an arithmetical or statistical comparison of values. Such statistical comparisons include, but are not limited to, applying a rule. When a deviation is found between phosphorylated ACC in platelets from a subject and a reference value, said deviation is indicative or conclusive that the status or condition of blood coagulation in the subject is different from that represented by the reference value. When (substantially) no deviation is found between phosphorylated ACC in platelets from a subject and a reference value, the absence of such deviation is indicative or conclusive that the status or condition of blood coagulation in the subject is substantially the same as that represented by the reference value.

When a deviation is found between phosphorylated ACC in platelets from a subject from two or more successive time points, said deviation is indicative or conclusive that the status or condition of blood coagulation in the subject has changed between the two or more successive time points.

When (substantially) no deviation is found between phosphorylated ACC in platelets from a subject from two or more successive time points, the absence of such deviation is indicative or conclusive that the status or condition of blood coagulation in the subject is substantially the same at the two or more successive time points.

A “deviation” of a value from a value to be compared with, e.g. a reference value, may generally encompass any direction (e.g., increase: value 1>value 2; or decrease: value 1<value 2) and any extent of alteration. For example, a deviation may encompass a decrease in a value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a reference value with which a comparison is being made. For example, a deviation may encompass an increase of a value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a reference value with which a comparison is being made. Preferably, a deviation may refer to a statistically significant observed alteration. For example, a deviation may refer to an observed alteration which falls outside of error margins of values in a given population (as expressed, for example, by standard deviation or standard error, or by a predetermined multiple thereof, e.g., ±1xSD or ±2xSD, or ±1xSE or ±2xSE). Deviation may also refer to a value falling outside of values in a given population (for example, outside of a range which comprises ≧40%, ≧50%, ≧60%, ≧70%, ≧75% or ≧80% or ≧85% or ≧90% or ≧95% or even ≧100% of values in said population). Alternatively, a deviation may be concluded if an observed alteration is beyond a given threshold or cut-off. Such threshold or cut-off may be selected as generally known in the art to provide for a chosen sensitivity and/or specificity of the methods described herein, e.g., sensitivity and/or specificity of at least 50%, or at least 60%, or at least 70%, or at least 80%, or at least 85%, or at least 90%, or at least 95%. Advantageously, steps involving comparison of phosphorylated ACC determined in platelets from a subject to a reference value or comparison of phosphorylated ACC determined in platelets from a subject from two or more successive time points with each other may be performed using a computer, such as a general-purpose computer. The hardware architecture of such a computer can be realised by a person skilled in the art, and may comprise hardware components including one or more processors (CPU), a memory (e.g., a random-access memory (RAM) and a read-only memory (ROM)), and an internal or external data storage medium (e.g., hard disk drive). A program configured to direct the computer to carry out the requisite computational steps may be provided on a computer-readable recording medium, such as, e.g., diskette, CD, DVD, etc.

By detecting a deviation between phosphorylated ACC in platelets from a subject and a reference value representing a given status or condition of blood coagulation in the subject, the uses and methods as taught herein may advantageously allow to detect a change (e.g., worsening or improvement) in the blood coagulation status of a subject before (other) symptoms or signs indicative of such change are felt or observed. Likewise, by detecting a deviation between phosphorylated ACC in platelets from a subject taken at different times, the uses and method as taught herein may advantageously allow to detect a change (e.g., worsening or improvement) in the blood coagulation status of a subject before (other) symptoms or signs indicative of such change are felt or observed. This early information about the blood coagulation status of the subject can allow to initiate therapeutic intervention(s) earlier and more effectively.

The methods and uses as taught herein also allow for monitoring the responsiveness of a subject to an anti-coagulant or pro-coagulant that has been administered to said subject and at the same time allow for determining the in vivo efficacy of said anti-coagulant or pro-coagulant.

Another application of the methods and uses as taught herein allows for evaluating the risk of bleeding after surgery. The methods and uses as taught herein allow to determine whether platelet activation and activation of the coagulation cascade are functional in bleeding patients. In addition, as shown in the experimental section, the level of ACC phosphorylation (directly linked to platelet activation and activation of the coagulation cascade) may be proportional to the importance of bleeding. Therefore, in a patient with normal coagulation, ACC phosphorylation may be an indicator of bleeding severity and predict complications.

Yet another application of the methods and uses as taught herein allow for evaluating the recovery of thrombin generation after surgery towards basal thrombin generation (i.e. thrombin generation before surgery). Thrombin generation can be quantified through measuring endogenous thrombin potential (ETP). As shown in the experimental section, the level of ACC phosphorylation may be proportional to the recovery of thrombin generation or ETP value after surgery.

Another aspect of the present invention relates to methods and uses for screening one or more test agents for candidate anti-coagulants or pro-coagulants.

As used herein, the term “agent” broadly refers to any chemical (e.g., inorganic or organic), biochemical or biological substance, molecule or macromolecule (e.g., biological macromolecule), a combination or mixture thereof, a sample of undetermined composition, or an extract made from biological materials such as bacteria, plants, fungi, or animal cells or tissues. Non-limiting “agents” include antibodies and fragments and derivatives thereof, polypeptides or proteins, peptides, peptidomimetics, aptamers, photoaptamers, chemical substances, preferably organic molecules, more preferably small organic molecules, lipids, carbohydrates, polysaccharides, nucleic acids, oligonucleotides, ribozymes, etc., and any combinations thereof.

The term “anti-coagulant” generally refers to an agent that prevents blood coagulation or delays blood coagulation, i.e. an agent that prevents blood from clotting or prevents a clot that has formed from enlarging. They may inhibit clot formation by blocking the action of clotting factors or platelets.

In preferred embodiments, the anti-coagulant administered to a subject or screened for in the methods and uses as taught herein is selected from the group comprising an inhibitor of thrombin production, an inhibitor of thrombin activity (e.g. a direct thrombin inhibitor), an inhibitor of binding of thrombin to a platelet thrombin receptor, a platelet thrombin receptor antagonist, inhibitor of platelet activation, preferably platelet thrombin receptor-mediated platelet activation, more preferably thrombin-induced platelet activation

The term “direct thrombin inhibitor” or “DTI” refers to an anticoagulant that directly inhibits thrombin.

The term “platelet thrombin receptor” as used herein generally refers to any receptor on the platelet surface to which thrombin binds and which activation by thrombin leads to an intracellular signalling. The term refers in particular to a platelet activated receptor (PAR), preferably human PAR-1 and PAR-4 and mouse PAR-3 and PAR-4. Suitable examples of a platelet thrombin receptor antagonist are E555 and Vorapaxar.

The term “pro-coagulant” generally refers to an agent that promotes blood coagulation.

In a preferred embodiment, the pro-coagulant administered to a subject or screened for in the methods and uses as taught herein is selected from the group comprising an activator of thrombin production or thrombin activity, thrombin, a platelet thrombin receptor agonist.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The above aspects and embodiments are further supported by the following non-limiting examples.

### Examples

### Example 1

### Experimental Procedures

### Animals

AMPK α1^{-/-} (mixed C57BL6 and SV-129 background) mice were generated as described (Jørgensen et al. 2004 J Biol Chem 279(2): 1070-1079). All animal procedures and protocols were approved by local authorities (Comité d'éthique facultaire pour I'expérimentation animale, UCL/MD/2007/049) and were performed in agreement with the guidelines on animal experimentation at our institution. Moreover, this study conforms to the Guide for the Care and Use of Laboratory Animals published by the US National Institutes of Health (NIH Publication No. 85-23, revised 1996).

### Study Population.

Blood was taken from 6 patients who underwent cardiac surgery with a cardio-pulmonary bypass procedure (CPB). None of these patients received aspirin or other anti-platelet agent at least 5 days before surgery. Patients who had suffered from acute coronary syndrome or other thrombotic processes in a period of 6 months before surgery, were excluded from the study.

### Study Design.

A blood sample was drawn during CPB. Under this condition, all patients received unfractionated heparin (UFH) as a bolus before CBP and had an activated coagulation time (ACT) above 450 s. ACT was measured at the bedside of the patients: a sample of blood was drawn, transferred into an appropriate test vial and the clotting time was measured. The second sample was taken 4 h after surgery and the coagulation was assessed in the same time (activated partial thromboplastin time, apTT; thrombin time, TT). Thrombin time (Thromboclotin™, Siemens, Marburg, Germany) and activated partial thromboplastin time (Platelin L; bioMerieux, France) were measured in a tube containing 0.129 M of trisodium citrate and determined using a coagulation device (MDA II coagulation analyzeer; bioMérieux). After surgery, a chest tube was inserted and a closed chest drainage system was attached to promote drainage of air and fluid. Bleeding was evaluated by the amount of blood collected in the chest tube of drainage over a period of 6 h after admission in intensive cares.

Endogenous thrombin potential (ETP) was measured using a fluorogenic thrombin generation assay. Briefly, 80 μl of platelet-poor plasma were pipetted into the well of a microplate together with 20 μl of a mixture of Tissue Factor (final concentration 20 μM) and phospholipids (final concentration 4 μM) (PPP-Reagent HIGH, Thrombinoscope by, Maastricht, The Netherlands). The plate was put in a pre-warmed measuring chamber and 20 μL of the fluorogenic substrate with CaCl₂ 0.1 M (FluCa Kit, Thrombinoscope by) was automatically dispensed into each well. The fluorescence signal was then recorded for 30 min at 15-second time intervals. As there is no direct correlation between thrombin activity and fluorescent signal intensity (amongst others because thrombin bound to α2-macroglobulin retains its activity to the fluorogenic substrate), the splitting of the fluorogenic substrate is compared to a constant known thrombin activity in a parallel non-clotting sample containing Thrombin Calibrator (Thrombinoscope by), the so called calibrated automated thrombogram. Accordingly, ETP values were calculated and corrected for α2-macroglobulin complex activity by using the Calibrated Automated

### Thrombogram.

Integrity of the platelet PAR was assessed by the Multiplate® TRAP test. Briefly, 300 μl of saline and 300 μl of a blood sample (usually hirudin or heparin blood) were pipetted into a Multiplate® test cell. The sample was allowed to warm and equilibrate for 3 minutes. Then, the activator thrombin receptor activating peptide (TRAP-6, a potent agonist which mimicks the platelet-activating action of thrombin) was added. The Multiplate® analyzer recorded platelet aggregation at approximately 0.5 second intervals. The principle of Multiplate® analysis is based on the fact that platelets become sticky upon activation and adhere and aggregate onto the metal sensor wires in the Multiplate® test cell. The increase in impedance by the attachment of activated platelets onto the Multiplate® sensors was transformed to arbitrary Aggregation Units (AU) and plotted against time. Three parameters were calculated: Aggregation, the Area Under the aggregation Curve (AUC) and Velocity. The most important parameter is AUC, which is recorded as Units (U). It is affected by the total height of the aggregation curve as well as by its slope and is best suited to express the overall platelet activity. The Aggregation (in AU) is the maximum height of the curve during the measurement period and the Velocity (in AU/min) is the maximum slope of the curve. The output data calculated by the software was the mean value of the two independent sensors in the test cell. The correlation coefficient of the individual measurements was determined and the analysis was accepted when the correlation coefficient was greater than 0.98. Additionally, the difference of each curve from the mean curve (DIF) was calculated and was accepted when the difference was less than 20%.

### Platelet Isolation

Washed human platelets (used in in vitro studies). Platelets were obtained from adult healthy volunteers in accordance with the Ethics Commission of our institution (Commission d'Ethique Biomédicale Hospitalo-Facultaire, Université catholique de Louvain). Whole blood was collected with butterfly needle 21G into Sodium Citrate 1:10 solution (S-monovette). Platelet-rich plasma (PRP) was obtained by 15 min centrifugation at 330 g. The platelets pellet was then washed in Tyrode modified buffer (135 mM NaCl, 12 mM NaHCO₃, 1 mM Sodium Citrate, 2.9 mM KCl, 0.3 mM Na₂HPO₄, 1 mM MgCl₂, 5 mM D-glucose, 10 mM Hepes, 0.35% bovine serum albumin (BSA), pH 7.4 at 37° C.) and resuspended to a density of 2.5 10⁵ platelets/μl for the measurement of intracellular Ca²⁺ or platelet aggregation. For Western Blot analysis, PRP with eptifibatide 4 μg/ml (Integriline, GlaxoSmithKline) and ectonucleotidase apyrase (grade I) 0.5 U/ml was layered over a density barrier of iodixanol (Optiprep®) that allowed the erythrocytes and leucocytes to pellet during centrifugation at 350 g for 15 min. The platelets, because of their small size, sediment much more slowly. After centrifugation at 800 g for 10 min, the resulting pellet was resuspended to a density of 2·10⁵ platelets/μl in Tyrode modified buffer before treatment and lysis of samples.

### Non-Washed Human Platelets (Used in In Vivo Studies).

Platelets were obtained from patients after admission in intensive cares, in accordance with the Ethics Commission of our institution. Whole blood was collected with butterfly needle 21G into Sodium Citrate 1:10 solution (S-monovette). PRP was obtained by 15 min centrifugation at 330 g. For Western Blot analysis, PRP with eptifibatide 4 μg/ml (Integriline, GlaxoSmithKline) and ectonucleotidase apyrase (grade I) 0.5 U/ml was layered over a density barrier of iodixanol (Optiprep®) that allowed the erythrocytes and leucocytes to pellet during centrifugation at 350 g for 15 min. The platelets, because of their small size, sediment much more slowly. After centrifugation at 800 g for 10 min, the resulting pellet was resuspended to a density of 2·10⁵ platelets/pi in Tyrode modified buffer before lysis of samples for Western blot analysis.

### Murine Platelets.

Eight- to 12-week-old mice were bled under sodium pentobarbital anesthesia (25-35 mg·kg⁻¹) from the retroorbital plexus. Mouse blood was collected in a 1/10 ACD solution (12 mM citric acid, 15 mM sodium citrate, 25 mM glucose) with ectonucleotidase apyrase (grade I) 0.5 U/ml. PRP was obtained by centrifugation at 800 g for 5 s followed by 5 min at 150 g. PRPs from three animals were pooled. Platelets were washed in Tyrode modified buffer and resuspended to a density of 2.5 10⁵ platelets/μl for the measurement of platelet aggregation and for Western Blot analysis. At least three independent experiments were performed on PRP from different pools.

### Measurement of Platelet Aggregation

Light transmission during thrombin-induced platelet aggregation in human and murine PRP was recorded with a Chrono-Log Aggregometer at 37° C., under stirring condition (1200 crpm). The platelet suspension was incubated with dimethyl sulfoxide (DMSO, vehicle) or STO-609 (10 μM, Tocris) for 2 min before the addition of the thrombin (+/−0.03 U/ml). CaCl₂ 2 mM was added prior to the agonists.

### Western Blotting

Proteins were separated as described (Miranda et al. 2010. J Clin Invest 103(6): 879-887). The membranes were probed with the following antibodies: α1/α2 AMPK (Kinasource), phospho-ACC (S79) (1:1000; Cell Signalling), phospho-MLC (S19) (1:10000; Sigma-Aldrich), phospho-VASP (T278) (1:1000; ECM Bioscience), cofilin (Cell Signalling), phospho-cofilin (Ser 3) (Cell Signalling). Bound antibodies were detected by enhanced chemiluminescence (Thermofisher). For each gel, control of loading was realized by immunoblotting using an anti-β-actin (1:10000; Santa Cruz Biotechnology), anti-gelsolin (Santa Cruz Biotechnology) or anti-GAPDH (1:1000; Cell Signalling) antibody after the membranes were stripped. Band intensities were quantified by scanning and processing image intensities with the program ImageJ (1.33 for Mac OS X). The quantification of immunoblots presented in results was obtained after normalization using the internal loading control.

### AMPK Enzyme Assays

AMPK was immunoprecipitated with 10 μg of α1-AMPK or α2-AMPK antibodies (Kinasource) from 50 μg of platelet extracts and assayed in a final volume of 50 μl Hepes 50 mM, pH 7.2, with 0.2 mM SAMS peptide (Substrate for AMP-activated Protein Kinase, Tocris Bioscience), 0.2 mM adenosine 5′-monophosphate (AMP), and 0.1 mM [³²P]MgATP (specific radioactivity: 1000 cpm/pmol; Perkin Elmer) at 30° C. Aliquots (10 μl) were removed and spotted onto Whatman P81 papers for measurement of ³²P incorporation. One unit of protein kinase activity corresponds to the incorporation of 1 nmol of phosphate into the appropriate peptide substrate per min under the assay conditions.

### Flow Cytometry

Flow cytometric analysis was performed on a FACScan (Becton Dickinson). Washed human platelets (2·10⁵ platelets/μl) were pre-incubated with DMSO/STO-609 for 2 min and then treated with or without thrombin (0.025 U/ml) in the presence of excessive amounts of FITC-conjugated monoclonal antibody (anti-CD62P (BD Biosciences) or PAC-1 (BD Biosciences)) at room temperature. The samples were then fixed at 4° C. for 30 min with 2% paraformaldehyde. The levels of P-selectin expression and PAC1 binding were expressed as the percentages of cells positive for anti-CD62P and PAC-1, respectively. The negative cut-off for each antibody was set using resting platelets that gave less than 5% of cells positive for binding of anti-CD62P or PAC-1.

### Cytosolic Ca²⁺ Measurement

Platelets pelleted from the platelet-rich plasma were suspended in Ca²⁺-free modified Tyrode's buffer and then incubated with 3 μM flou-3/acetoxymethyl ester (Santa Cruz Biotechnology) at 37° C. for 30 min. Probenecid 2.5 mM (Sigma-Aldrich) was added to the buffer throughout the experiments to prevent leakage of the dye. After 2 washings, fluo-3-loaded platelets were suspended in modified Tyrode's buffer at a concentration of 2 10⁷ platelets/ml. They were then pre-incubated with 10 μM STO-609 in the presence of 1 mM extracellular Ca²⁺ with stirring at 37° C. for 2 min before the addition of thrombin (0.1 U/ml). Fluorescence (excitation 505 nm, emission 530 nm) was measured with a spectrophotometer (AMINCO SPF500). Ca²⁺ concentration as estimated as follows: Kd* (F−Fmin)/(Fmax−Fmin), where the Kd of Fluo-3 is (864 mM at 37° C.). The minimal fluorescence (Fmin) is the fluorescence in the absence of Ca²⁺ and Fmax is the fluorescence determined with ionomycin 1 μM.

### Thromboxane B2 Assay

Because thromboxane A2 is very unstable and converts rapidly to TXB2, a more stable metabolite, the latter was measured. After 2 min pre-incubation with 10 μM STO-609 or DMSO, washed platelets were treated with 0.1 U/ml thrombin for 5 min. Negative controls corresponded to platelets activated by thrombin in the presence of 1 mM acetylsalicylic acid (ASA). Platelet suspensions were pelleted (quick speed), and TXB2 in the supernatants was assayed according to the manufacturers instructions (Enzo Life Sciences).

### Example 2

### Thrombin Induces AMPK Activation In Vitro Via the Stimulation of CaMKKβ

Resting platelets were purified from human peripheral blood as described in the experimental procedures.

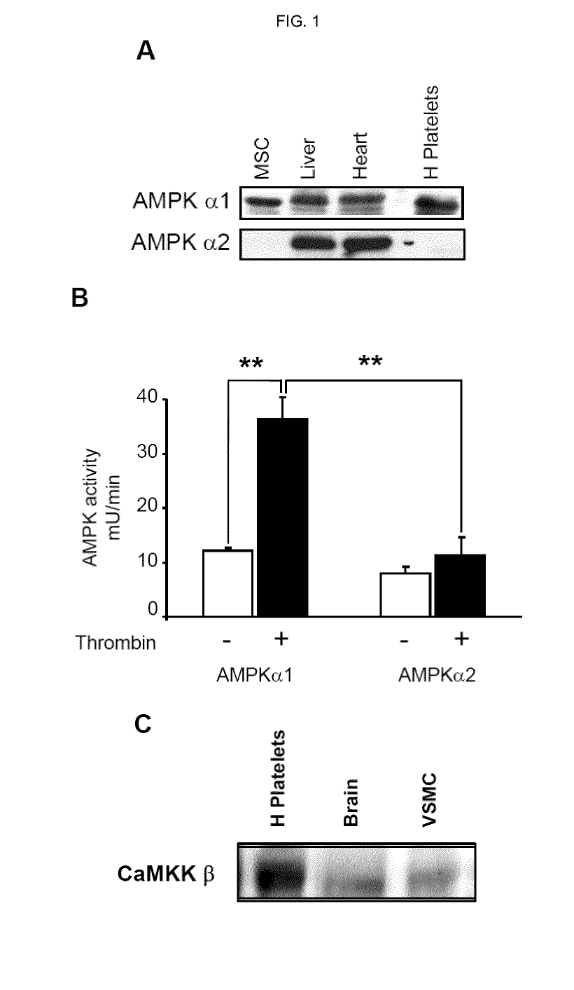

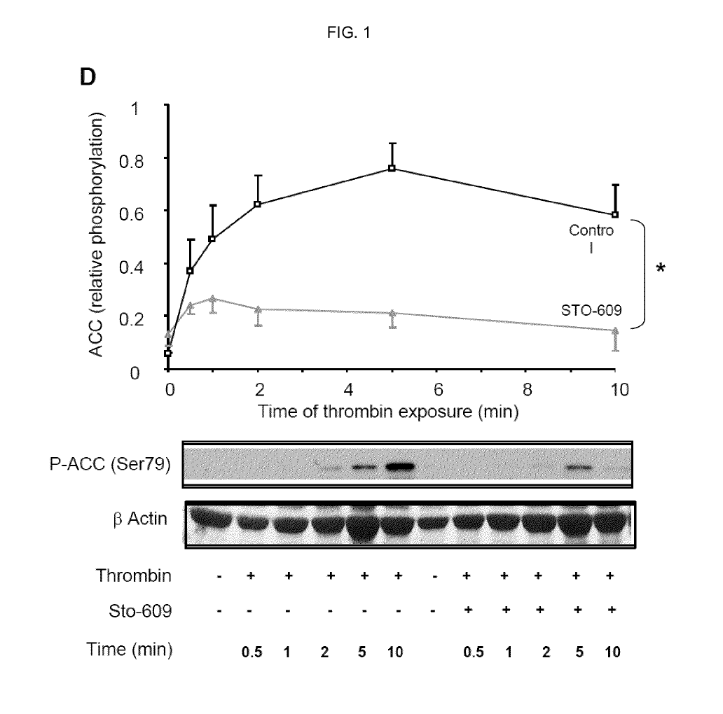

AMPK expression in human platelets was analysed by Western Blotting. FIG. 1A shows that human platelets expressed the α1 subunit of AMPK. Extracts from mesenchymal stem cells (MSC) and from liver and heart were used as a positive control for α1 and α2 expression, respectively.

Treatment of the human platelets with 0.1 U/ml thrombin led to AMPK activation (FIG. 1B). AMPK was immunoprecipitated with anti-α1- and anti-α2-AMPK antibodies prior to AMPK assay. The AMPK catalytic α1-subunit isoform accounted for the total AMPK activity in platelets (FIG. 1B), confirming the immunoblotting results.

Thrombin-induced AMPK activation was mediated by CaMKKβ, an upstream kinase in the AMPK cascade (FIG. 1D). Human platelets were shown to express the CaMKKβ1 isoform predominantly expressed in rat brain and in vascular smooth muscle cells (VSMC) by probing platelet extracts with a polyclonal antibody that recognizes CaMKKβ. The platelets extracts contained a major band (65,000) corresponding to the CaMKKβ1 isoform (FIG. 1C).

STO-609 (Tocris) is a relatively selective and cell permeable inhibitor of CaMKKβ. At a concentration of 10 μM, STO-609 markedly reduced AMPK activation, as judged by a decrease in phosphorylation of AMPK Thr172 (not shown) and of downstream Ser79 acetyl-CoA carboxylase (ACC) (FIG. 1D).

### Example 3

### STO-609 Prevents Platelet Aggregation and Secretion In Vitro

To evaluate the potential implication of CaMKKβ in platelet aggregation, washed human platelets were pre-incubated for 2 min with 10 μM STO-609, prior to treatment with thrombin or ADP.

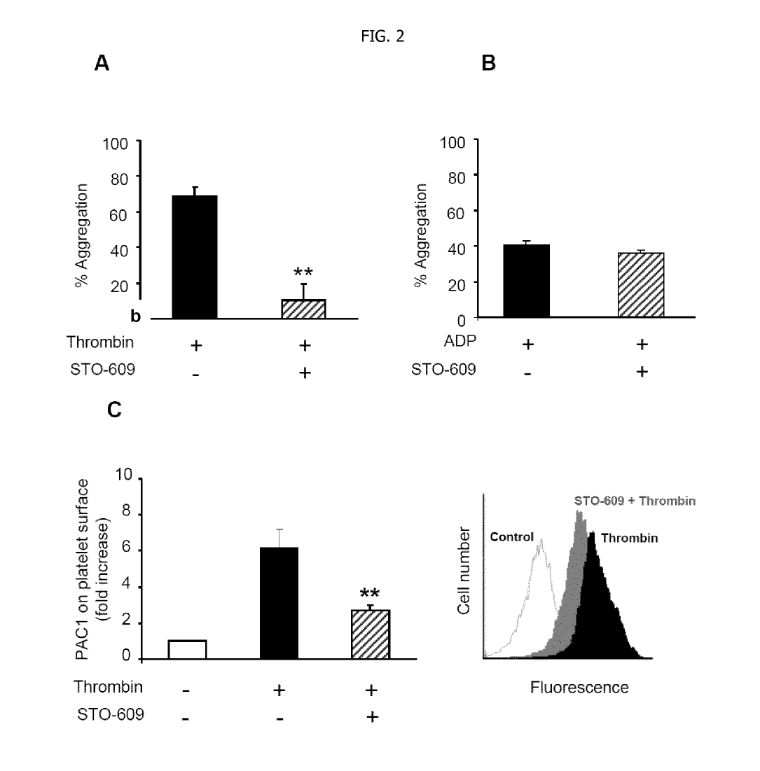

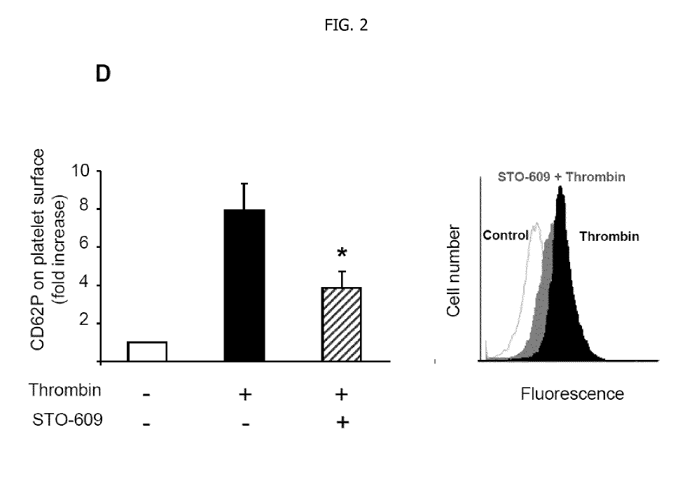

STO-609 significantly inhibited thrombin-induced platelet aggregation (0.025 U/ml thrombin: 69±3% of aggregation; thrombin+ST0609: 11±4% of aggregation, P<0.001) (FIG. 2A).

Platelet aggregation caused by 20 μM ADP was not inhibited by STO-609 (FIG. 2B). ADP did also not lead to ACC phosphorylation (not shown), indicating that the CaMKKβ/AMPK pathway is specifically activated by thrombin.

In addition to aggregation measurements, we examined by flow cytometry STO-609 effects on PAC-1 binding, an antibody specific for the activated conformation of αIIbβ3. Platelets were activated by 0.025 U/ml thrombin in the absence or presence of 10 μM STO-609. The CaMKKβ inhibitor STO-609 significantly inhibited PAC-1 binding in response to thrombin (FIG. 2C).

In addition, the effect of STO-609 on α-granule release was determined by measuring the exposure on platelet surface of P-selectin (CD62P). STO-609 significantly inhibited CD62P detection per platelet in response to thrombin treatment (FIG. 2D).

### Example 4

### STO-609 Prevents MLC and VASP Phosphorylation In Vitro

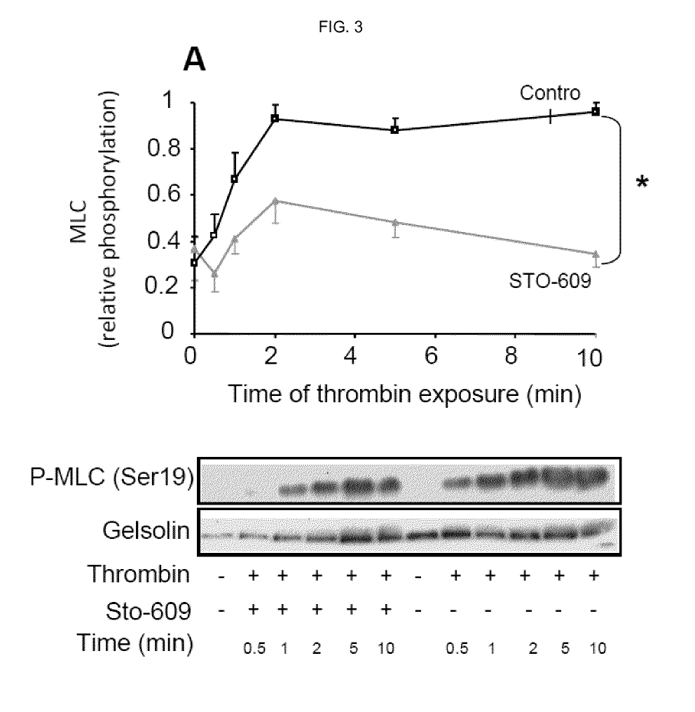

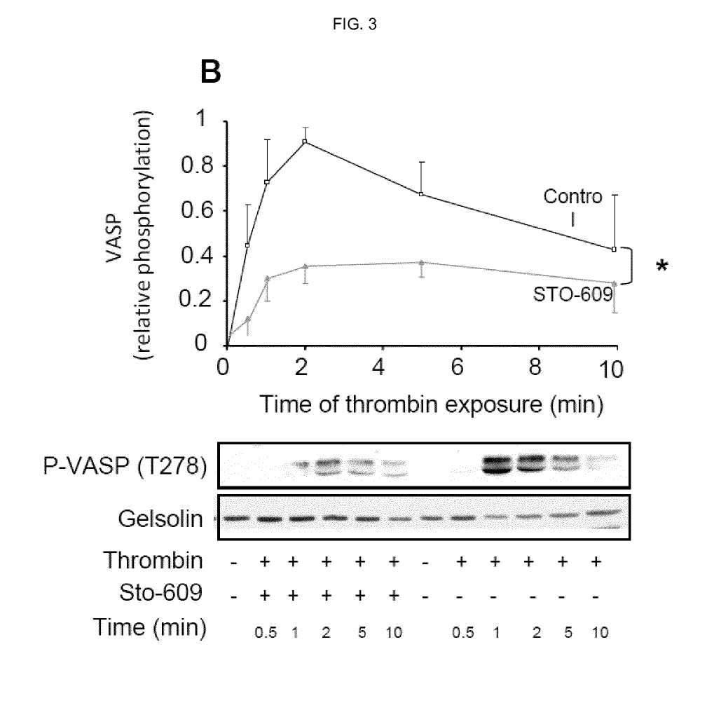

Human platelet incubation with thrombin led to an increase in myosin light chain (MLC) Ser 19 phosphorylation (FIG. 3A), which was significant after 2 min and persisted till 10 min of treatment. Pre-incubation with STO-609 decreased significantly MLC phosphorylation (FIG. 3A).

In human platelets, thrombin led also to a significant increased phosphorylation of VASP on Thr 278 (FIG. 3B). Pre-incubation with STO-609 prevented thrombin-induced VASP phosphorylation on this site (FIG. 3B).

These data show that the CaMKKβ/AMPK cascade is involved in MLC and VASP phosphorylation and suggest that the CaMKKβ/AMPK cascade may play a role in the changes in actin cytoskeleton induced by thrombin in platelets.

### Example 5

### Platelet Aggregation and MLC Phosphorylation is Decreased in AMPK α1-Knockout Mice

As the α1-subunit isoform accounted for the total AMPK in human platelets (FIG. 1A,B), we used α1-AMPK knockout (KO) mice and we examined thrombin-induced platelet aggregation and MLC phosphorylation.

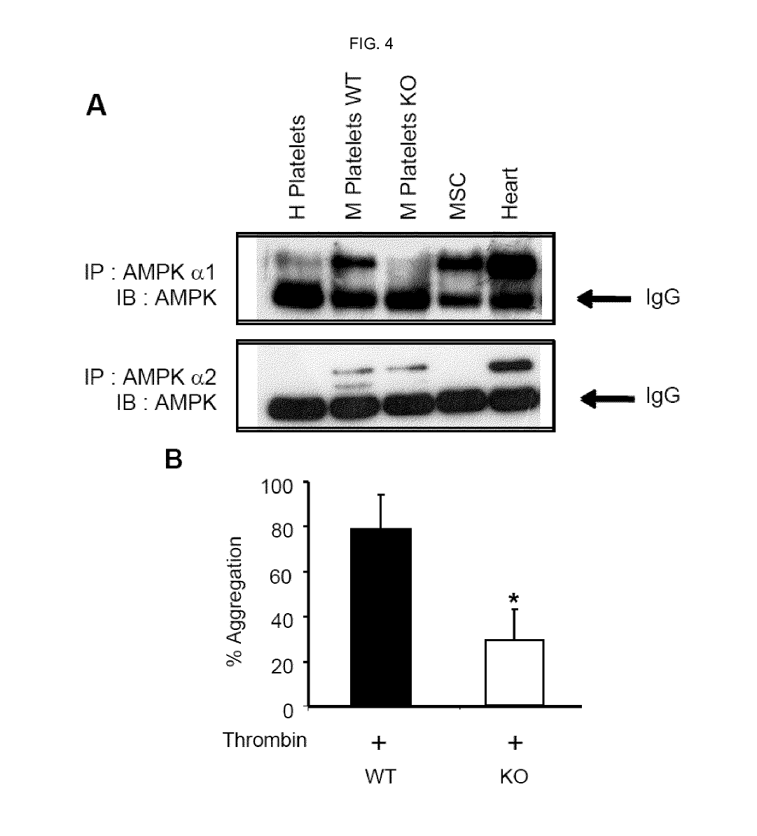

By contrast with human platelets, platelets from wild-type (WT) mice expressed both α-AMPK isoforms (FIG. 4A). As expected, no α1-AMPK could be detected from α1-AMPK KO mice.

In agreement with the data obtained using human platelets, thrombin-induced aggregation was attenuated in platelets from KO mice versus WT mice (FIG. 4B).

### Example 6

### Analysis of Blood Coagulation in Patients Undergoing Cardiac Surgery

During cardiac surgery, each of 6 patients received unfractionated heparin (UFH) to reach an activated coagulation time (ACT) above 450 sec. The mean ACT during the first sampling was 568±65 sec after administration of 25250±11000 UI UFH (Table 7). Under these conditions, activated partial thromboplastin time (aPTT) and thrombin time (TT) values were above 180 sec and 120 sec, respectively.

The second sample was taken 4 h after the admission in intensive cares. A coagulation assessment was performed in the same time. At this time, aPTT was 30±2 sec and TT was 25±3 sec, demonstrating a normal capacity to coagulate (Table 7). The International Normalized Ratio (INR) and fibrinogen were normal (data not shown) and platelets count was above 100 000/mm³ for each patient.

TABLE 1
Assessment of blood coagulation in patient 1. Patient 1
underwent aortic valve replacement. Blood coagulation
was assessed during and 4 h after surgery.

| | during surgery | post-surgery |
|---|---|---|
| ACT (s) | 485 | not applicable |
| aPTT (s) | >180 | 34 |
| TT (s) | >120 | 21 |
| UFH (units) | 17000 | — |
| chest tube drainage over 6 h | — | 165 |

TABLE 2
Assessment of blood coagulation in patient 2.
Patient 2 underwent Bentall operation.
Blood coagulation was assessed during and 4 h after surgery.

| | during surgery | post-surgery |
|---|---|---|
| ACT (s) | 523 | not applicable |
| aPTT (s) | >180 | 32 |
| TT (s) | >120 | 23 |
| UFH (units) | 33000 | — |
| chest tube drainage over 6 h | — | 160 |

TABLE 3
Assessment of blood coagulation in patient 3.
Patient 3 underwent mitral valvuloplasty.
Blood coagulation was assessed during and 4 h after surgery.

| | during surgery | post-surgery |
|---|---|---|
| ACT (s) | 635 | not applicable |
| aPTT (s) | >180 | 30 |
| TT (s) | >120 | 28 |
| UFH (units) | 25500 | — |
| chest tube drainage over 6 h | — | 80 |

TABLE 4
Assessment of blood coagulation in patient 4.
Patient 4 underwent Tirone-David operation.
Blood coagulation was assessed during and 4 h after surgery.

| | during surgery | post-surgery |
|---|---|---|
| ACT (s) | 498 | not applicable |
| aPTT (s) | >180 | 29 |
| TT (s) | >120 | 24 |
| UFH (units) | 10000 | — |
| chest tube drainage over 6 h | — | 105 |

TABLE 5
Assessment of blood coagulation in patient 5. Patient
5 underwent aortic valve replacement. Blood coagulation
was assessed during and 4 h after surgery.

| | during surgery | post-surgery |
|---|---|---|
| ACT (s) | 698 | not applicable |
| aPTT (s) | >180 | 30 |
| TT (s) | >120 | 24 |
| UFH (units) | 25000 | — |
| chest tube drainage over 6 h | — | 335 |

TABLE 6
Assessment of blood coagulation in patient 6. Patient
6 underwent CABG. Blood coagulation was
assessed during and 4 h after surgery.

| | during | admission in | after protamine |
|---|---|---|---|
| | surgery | intensive care unit | injection |
| ACT (s) | 568 | not applicable | not applicable |
| aPTT (s) | >180 | 40 | 27 |
| TT (s) | >120 | >120 | 30 |
| UFH (units) | 41000 | — | — |
| chest tube drainage | — | 520 | |
| over 6 h | | | |

TABLE 7
Assessment of blood coagulation in patients undergoing and
recovering from cardiac surgery. Mean values for the blood
coagulation parameters obtained from patients 1-6.

| | during surgery | post-surgery |
|---|---|---|
| ACT (s) | 568 ± 65 | not applicable |
| aPTT (s) | >180 | 30 ± 2 |
| TT (s) | >120 | 25 ± 3 |

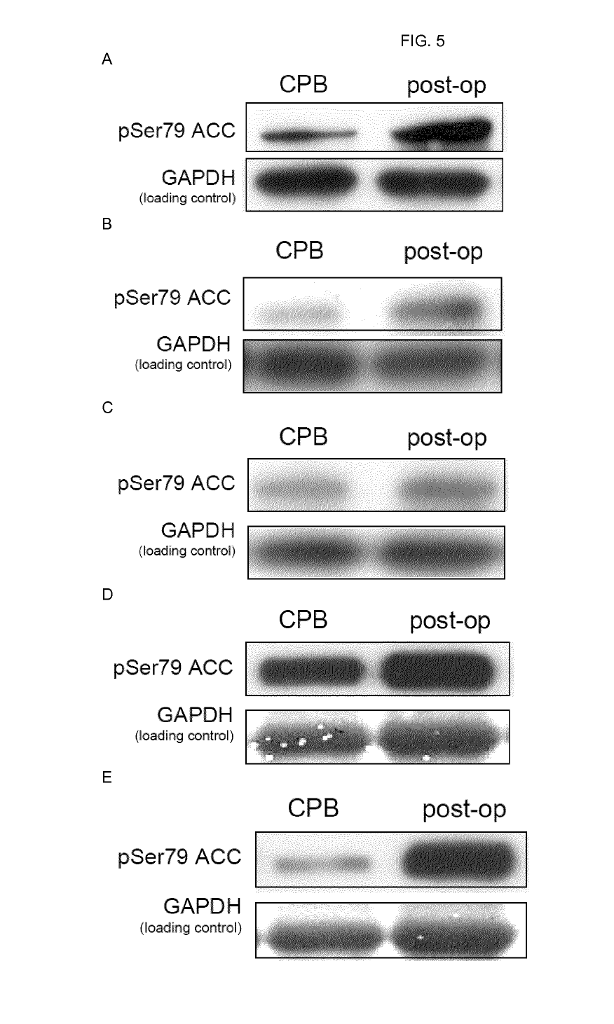

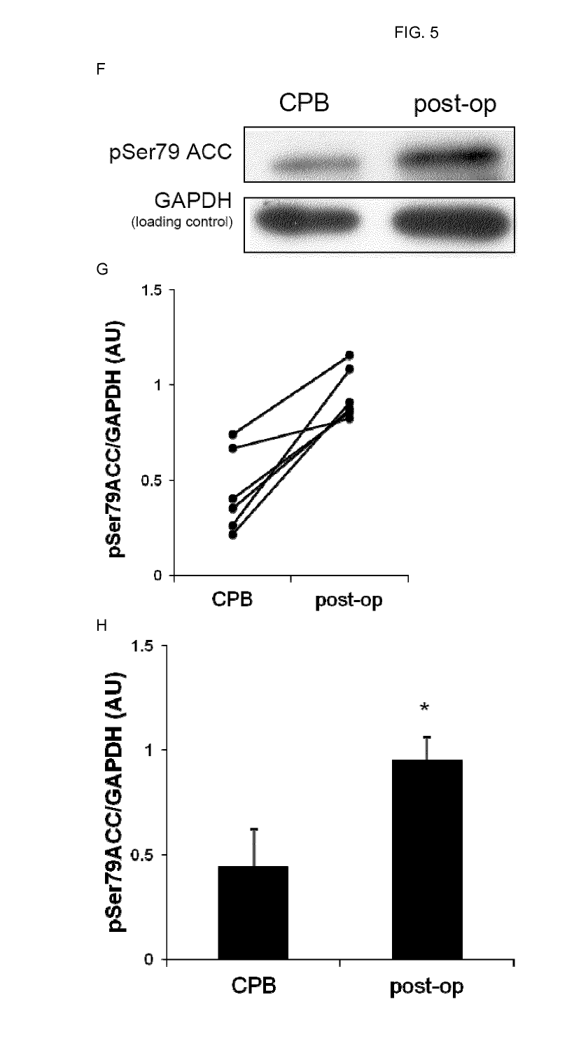

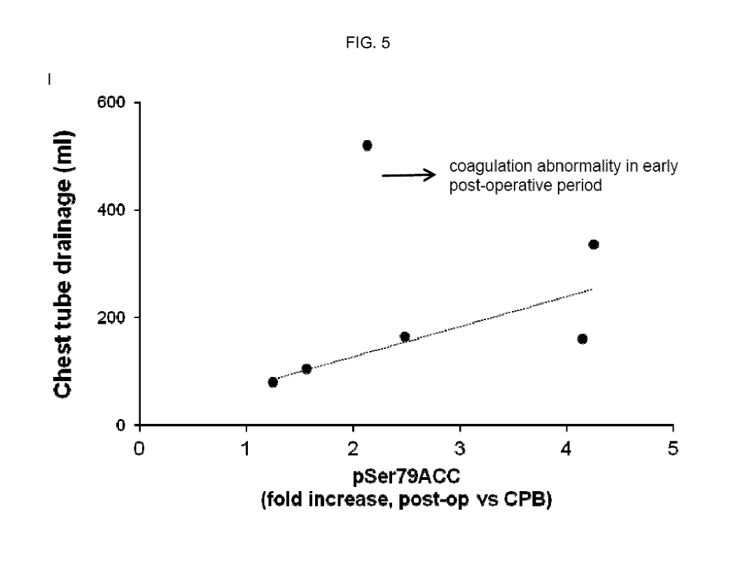

ACC phosphorylation significantly increased in the post-operative status (with normal coagulation) compared to the CPB status (under high dose of UFH) (FIG. 5H). Each patient individually exhibited an increase in ACC phosphorylation (FIGS. 5A-F and 6A). However, the extent of the effect varied between subjects. We correlated this effect with the post-operative bleeding severity characterized by chest tube drainage and showed that the bleeding severity was proportional to the increase in ACC phosphorylation (FIG. 5I). Note that bleeding for the patient 6 was more pronounced than expected. By contrast with the other patients, he exhibited an abnormal coagulation immediately after the admission in the intensive cares, which was responsible for a more important bleeding during this initial phase. Bleeding was controlled after restoration of coagulation by protamine administration.

In conclusion, our data clearly indicate that phosphorylation of ACC reflects coagulation cascade activation. It could predict bleeding complications after major surgery in patients with normal coagulation.

### Example 7

### Analysis of Blood Coagulation in Patients Undergoing Cardiac Surgery (Larger Study)

A larger study was set up to evaluate blood coagulation in a clinical situation in accordance with the study design of Examples 1 and 6. 29 patients who underwent elective cardiac surgery with CPB were included in the study. Diabetic patients were excluded from the study.

Blood samples were drawn before the surgery (basal); during surgery, after bolus administration of unfractionated heparin (UFH) to reach an activated clotting time (ACT) above 450 s; and 4 hours after surgery after blood coagulation correction (post-surgery).

Blood coagulation was assessed during surgery and post-surgery through measurement of activated partial thromboplastin time (aPPT) and thrombin time (TT).

During surgery, the mean ACT was 594±100 sec after administration of 47086±17766 UI UFH (Table 8). Under these conditions, activated partial thromboplastin time (aPTT) and thrombin time (TT) values were above 180 sec and 120 sec, respectively. The second blood coagulation assessment was performed 4 hours after surgery. At this time, aPTT was 36.3±7.4 sec and TT was 21.3±5.2 sec, demonstrating a normal capacity to coagulate (Table 8). The International Normalized Ratio (INR) and fibrinogen were normal (data not shown) and platelets count was above 100 000/mm³ for each patient.

TABLE 8
Assessment of blood coagulation in patients undergoing and
recovering from cardiac surgery. Mean values for the blood
coagulation parameters obtained from the patients in the study (n = 29).

| | during surgery | post-surgery |
|---|---|---|
| ACT (s) | 594 ± 100 | not applicable |
| aPTT (s) | >180 | 36.3 ± 7.4 |
| TT (s) | >120 | 21.3 ± 5.2 |

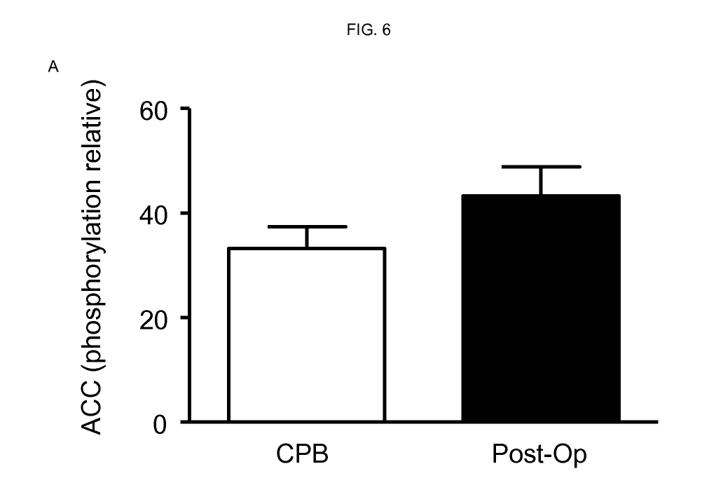

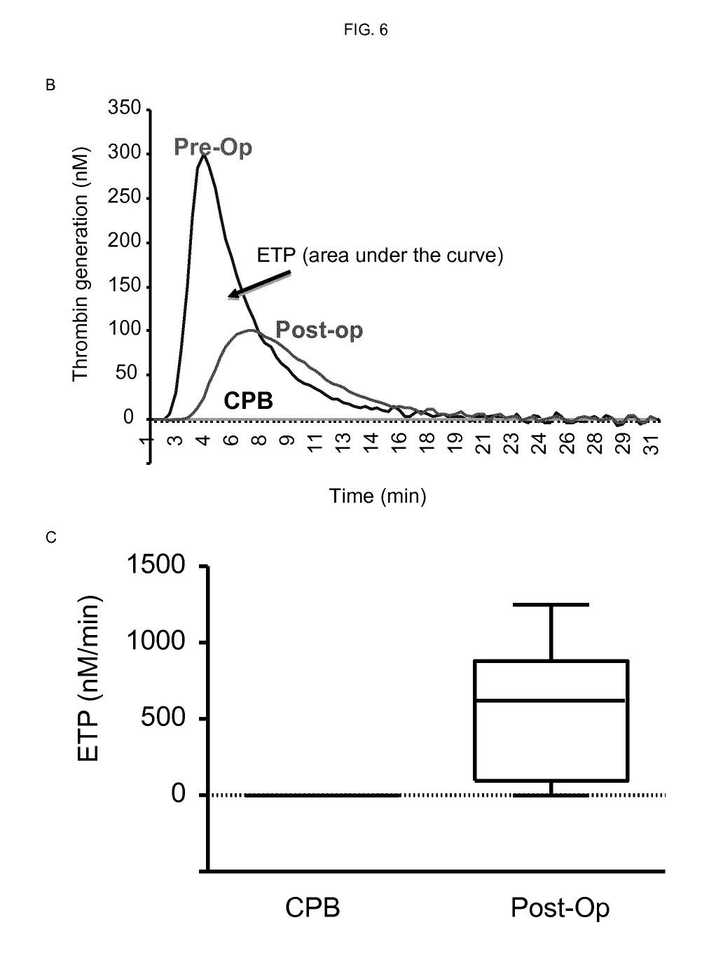

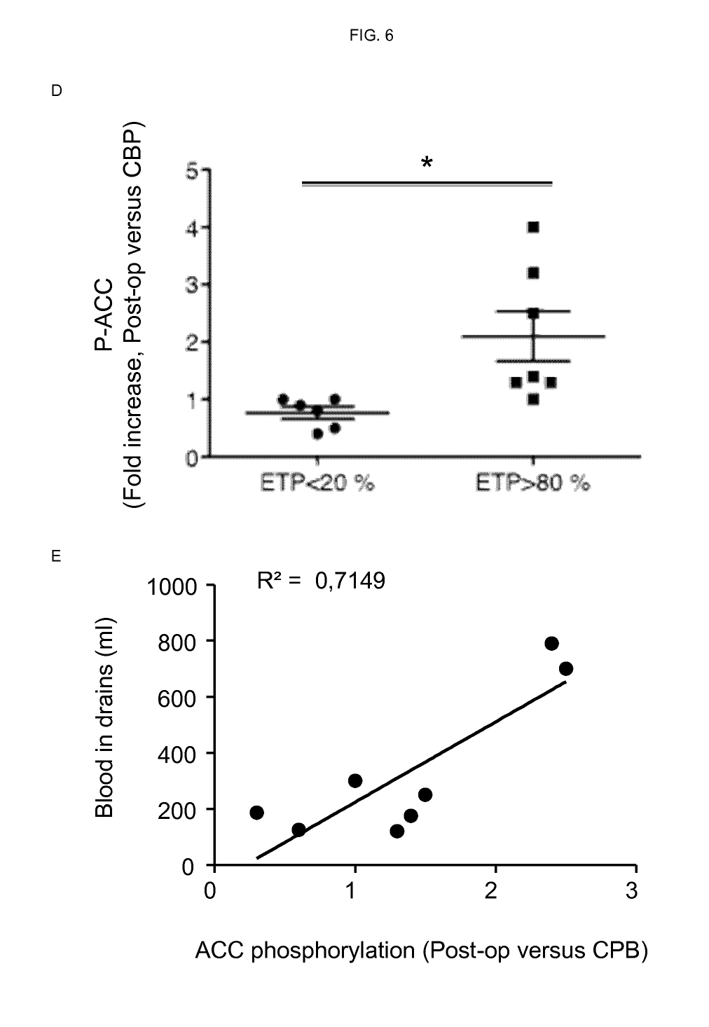

Platelets were isolated from the blood samples drawn during surgery and after surgery and ACC phosphorylation was evaluated. As shown in FIG. 6A, ACC phosphorylation significantly increased in the post-operative status (with normal coagulation) as compared to the CPB status (under high dose of UFH).

Thrombin generation was assessed through measurement of endogeneous thrombin potential (ETP) before surgery, during surgery and 4 hours after surgery. Following cardiac surgery, there is a partial recovery of basal (i.e. before surgery) thrombin generation (FIG. 6B,C). The huge heterogeneity of ETP values observed post-surgery reflects the random ability of patients to generate thrombin after CPB (FIG. 6C).

ACC phosphorylation was evaluated in 2 extreme groups of patients, recovering more than 80% and less than 20% of basal ETP, respectively. The increase in ACC phosphorylation in the post-operative status as compared to the CPB status was significantly higher in the platelets of patients preserving an intact ability to generate thrombin (ETP>80%) (FIG. 6D). These data support the use of phosphorylated ACC in platelets from a subject as a biomarker for thrombin generation in vivo (i.e. in said subject).

The integrity of the platelet PAR receptor, and its ability to be activated upon thrombin stimulation were evaluated using the Multiplate® TRAP test.

The increase in ACC phosphorylation in the post-operative status as compared to the CPB status was correlated with the post-operative bleeding severity characterized by chest tube drainage in patients with a positive TRAP test (≧830 A.U.) and without transfusion of platelets during CPB. The bleeding severity was proportional to the increase in ACC phosphorylation in these patients (R²=0.7149; FIG. 6E). These data support the use of phosphorylated ACC in platelets from a subject as a biomarker for platelet responsiveness, in particular thrombin-induced platelet responsiveness, in said subject.

In conclusion, our data indicate that phosphorylated ACC in platelets from a patient can be simultaneously used as a biomarker of thrombin generation and platelet responsiveness in said patient.

### Example 8

### Thrombin Specifically Induces ACC Phosphorylation in Human Platelets

Platelets were isolated from human blood samples as described in the experimental procedures. The isolated platelets were treated with increasing doses of thrombin (0.01, 0.03, 0.1, 0.3 and 1 U/ml), the thromboxane A2 (TXA2) receptor agonist U46619 (0.1, 0.3, 1, 3 and 10 μM), collagen (0.5, 1, 2.5, 5 and 10 μg/ml) or ADP (2.5, 5, 10, 25 nd 50 μM) for 1 min. Following treatment, the platelets were lysed and subjected to Western blotting for determining phosphorylation of Ser79 of ACC.

Thrombin induced Ser79 ACC phosphorylation, confirming the results of Example 2. The other platelet agonists ADP, collagen and U46619 failed to increase ACC phosphorylation comparably to thrombin (FIG. 7).

These data indicate that phosphorylated ACC in platelets may be useful as biomarker for blood coagulation specifically mediated by thrombin.

### Example 9

### The CaMKKβ Inhibitor STO-609 does not Interfere with Ca²⁺ Signalling or Thromboxane B2 (TXB2) Formation in Human Platelets

To evaluate the potential implication of CaMKKβ in Ca²⁺ signalling and thromboxane B2 (TXB2) formation, platelets were pre-incubated with STO-609 prior to treatment with thrombin. Cytosolic Ca²⁺ and thromboxane B2 (TXB2) formation were measured as described in the experimental procedures.

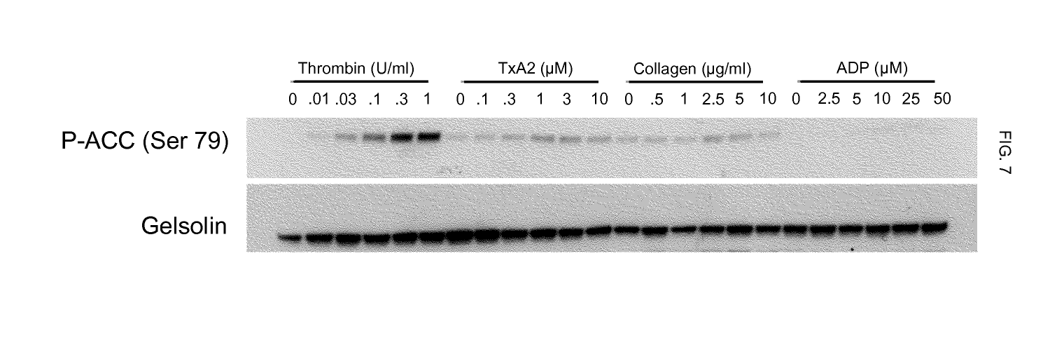

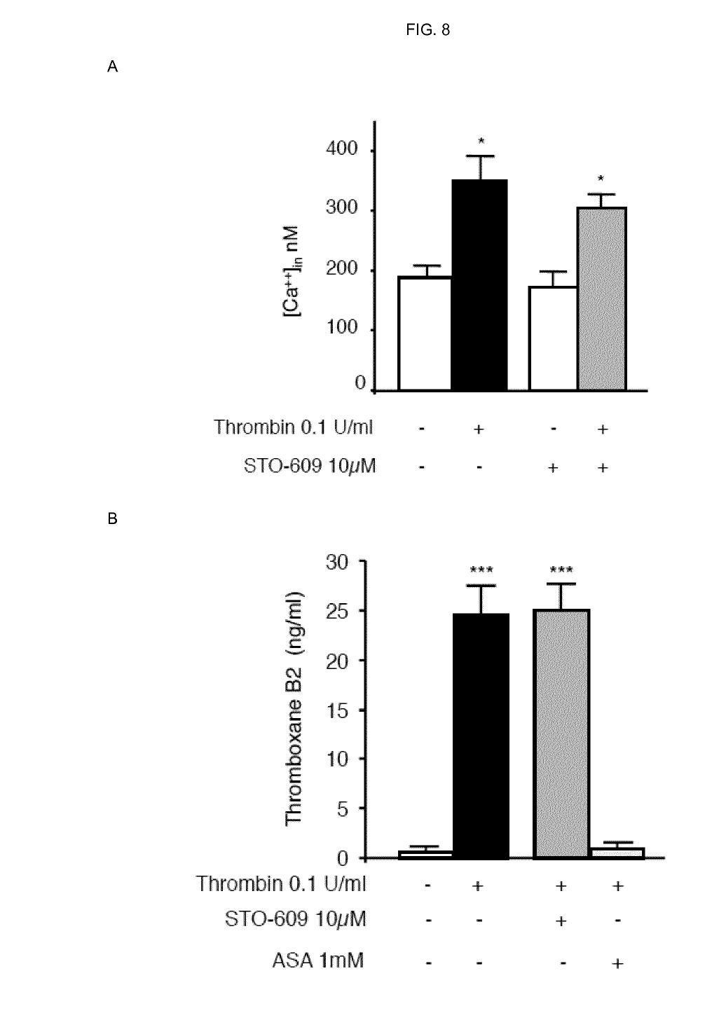

STO-609 did not modify the rise in Ca²⁺ concentration in response to thrombin (FIG. 8A). STO-609 did also not affect thrombin-induced TXB2 formation (FIG. 8B).

These data show that the inhibitory effect of STO-609 on platelet activation, aggregation and degranulation as shown in Example 3, did not result from alteration of Ca²⁺ signalling or thromboxane B2 (TXB2) formation.

### Example 10

### STO-609 Prevents Thrombin-Induced Cofilin Phosphorylation In Vitro

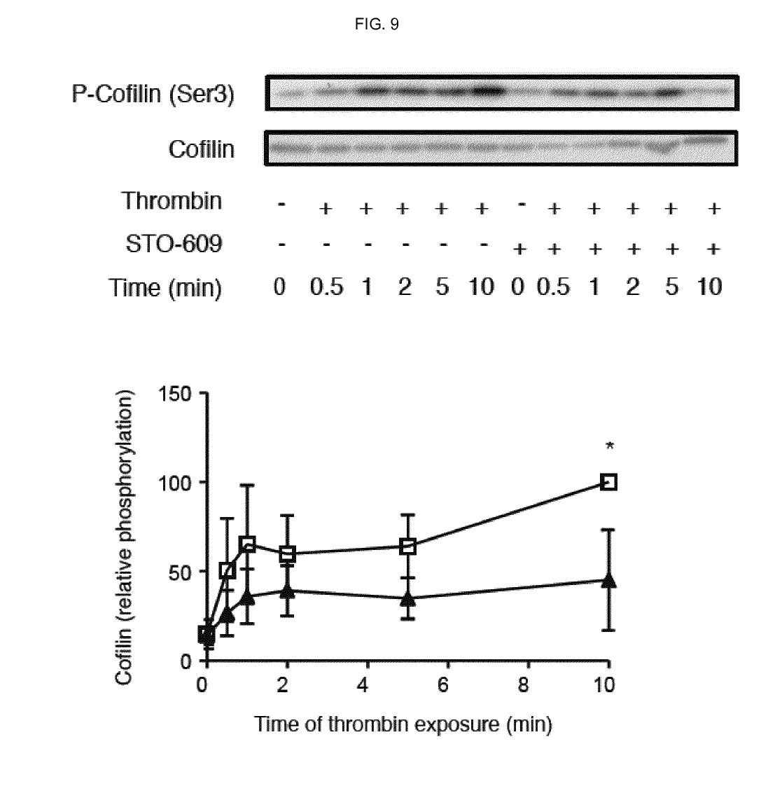

Human platelet incubation with thrombin led to an increase in cofilin phosphorylation in Ser3, which was maximal at 1 min and persisted for 10 min (FIG. 9). Pre-incubation with STO-609 significantly decreased cofilin phosphorylation 10 min after thrombin exposure (FIG. 9).

These data show that the CaMKKβ/AMPK cascade is involved in cofilin phosphorylation and suggest that the CaMKKβ/AMPK cascade may play a role in the changes in actin cytoskeleton induced by thrombin in platelets.

### Example 11

### ACC, MLC, VASP and Cofilin Phosphorylation are Decreased in AMPK α1-Knockout Mice

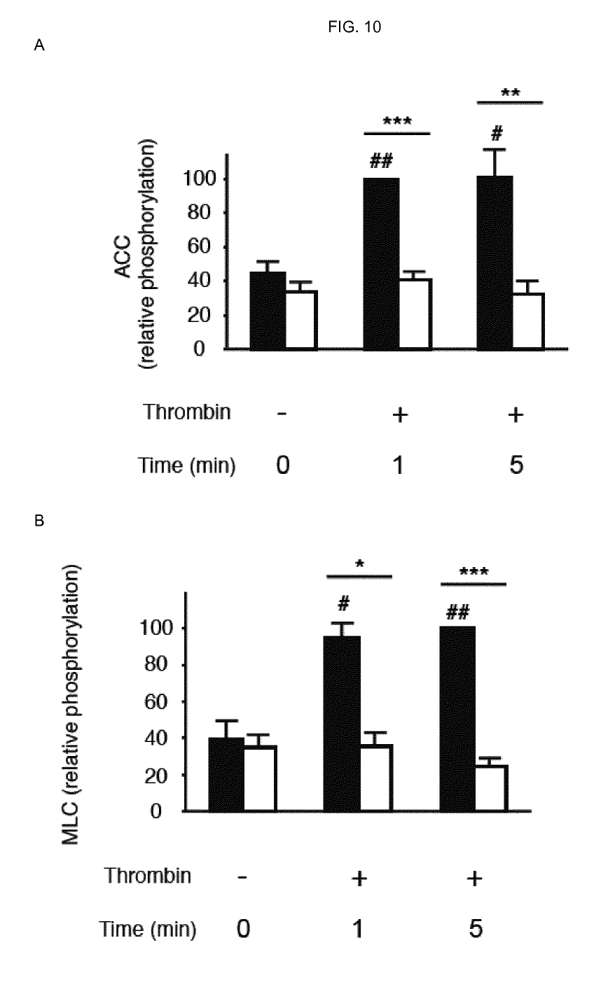

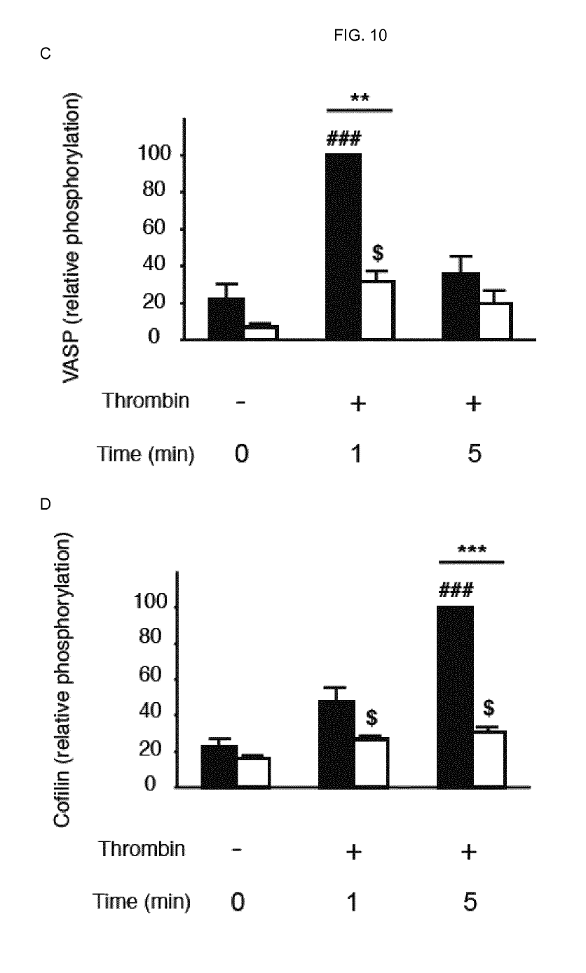

Platelets isolated from α1-AMPK knockout (KO) mice were used to examine the role of this AMPK isoform in thrombin-induced ACC, MLC, VASP and cofilin phosphorylation. The increase in ACC phosphorylation was reduced by about 75% in platelets from KO compared to WT mice. The residual phosphorylation may result from AMPK-α2 activation (FIG. 10A).

Thrombin 0.05 U/ml induced an increase in the phosphorylation of MLC, VASP and cofilin in platelets isolated from WT mice (FIG. 10B-D). The phosphorylation of these cytoskeleton proteins was significantly reduced in platelets from their KO littermates (FIG. 10B-D). These data indicate that MLC, VASP and cofilin are substrates of AMPK-α1 in murine platelets.

In agreement with the data obtained using human platelets, thrombin-induced ACC, MLC, VASP and cofilin phosphorylation were decreased in platelets from KO mice versus WT mice.

1. A method of assessing platelet and coagulation cascade activation comprising measuring phosphorylated Acetyl-CoA carboxylase (ACC) in platelets from a subject.

2. The method according to claim 1, further comprising comparing the measured phosphorylated ACC in platelets with a reference value to diagnose or monitor blood coagulation in the subject.

3. A method for determining blood coagulation in a subject comprising determining phosphorylated ACC in platelets from the subject.

4. The method according to claim 3, comprising:
(i) determining phosphorylated ACC in platelets from the subject;
(ii) comparing phosphorylated ACC as determined in (i) with a reference value, said reference value representing a known status of blood coagulation;
(iii) finding a deviation or no deviation of phosphorylated ACC as determined in (i) from said reference value; and
(iv) attributing said finding of deviation or no deviation to a particular diagnosis of blood coagulation in the subject.

5. The method according to claim 4, wherein in step (iv) higher phosphorylated ACC in platelets from the subject as compared to the reference value is attributed to increased blood coagulation compared to the blood coagulation status represented by the reference value.

6. The method according to claim 3, comprising:
(i) determining phosphorylated ACC in platelets from the subject from two or more successive time points;
(ii) comparing said phosphorylated ACC as determined in (i);
(iii) finding a deviation or no deviation of said phosphorylated ACC as compared in (b);
(iv) attributing said finding of deviation or no deviation to a particular change in blood coagulation in the subject between the two or more successive time points.

7. The method according to claim 3, wherein said blood coagulation comprises thrombin production.

8. The method according to claim 3, wherein said blood coagulation comprises the activation of a platelet thrombin receptor.

9. The method according to claim 3, wherein said blood coagulation comprises platelet activation.

10. The method according to claim 9, wherein said platelet activation results in platelet aggregation and/or platelet degranulation.

11. The method according to claim 3, wherein said subject has been administered an anti-coagulant or a pro-coagulant.

12. The method according to claim 11, wherein said subject has been administered an anti-coagulant, said anti-coagulant is an inhibitor of thrombin production, an inhibitor of thrombin activity, an inhibitor of binding of thrombin to a platelet thrombin receptor, a platelet thrombin receptor antagonist, or an inhibitor of platelet activation.

13. The method according to claim 3, wherein said subject is a patient with a blood coagulation disorder.

14. (canceled)

15. A method for screening one or more test agents for a candidate anti-coagulant or pro-coagulant, comprising determining whether phosphorylated ACC is modulated in platelets from a subject, wherein the subject has been administered the test agent.

16. A method for screening one or more test agents for a candidate anti-coagulant or pro-coagulant comprising:
(i) contacting a test agent with platelets;
(ii) determining whether said test agent can modulate phosphorylated ACC in said platelets.

17. The method according to claim 15, wherein said anti-coagulant is an inhibitor of thrombin production, an inhibitor of thrombin activity, an inhibitor of binding of thrombin to a platelet thrombin receptor, a platelet thrombin receptor antagonist, or an inhibitor of platelet activation.

18. (canceled)

19. A method for determining thrombin production, activation of a platelet thrombin receptor, or platelet activation, or platelet aggregation and/or platelet degranulation, comprising determining phosphorylated ACC in platelets from a subject.

20. The method according to claim 3, wherein the phosphorylated ACC is determined by measuring the levels of phosphorylated ACC is in the platelets from the subject.

21. The methods according to claim 8, wherein the blood coagulation is PAR.

22. The method of claim 21, wherein the PAR is PARI, PAR4, or combinations thereof.

23. The method according to claim 9, wherein the platelet activation is platelet thrombin receptor-mediated platelet activation or thrombin-induced platelet activation.

24. The method according to claim 12, wherein the inhibitor of thrombin activity is a direct thrombin inhibitor.

25. The method according to claim 12, wherein the inhibitor of platelet activation is an activator of thrombin production or activity, thrombin, or a platelet thrombin receptor agonist.

26. The method according to claim 4, further comprising administering to the subject an anti-coagulant or a pro-coagulant based on the finding of deviation relative to the said reference value.
